(19) United States
(12) Patent Application Publication
(10) Pub. No.: US 2025/0241999 A1
(43) Pub. Date: Jul. 31, 2025

(54) DELIVERY OF THERAPEUTIC RECOMBINANT URICASE USING NANOPARTICLES

(71) Applicants: Georgia State University Research Foundation, Inc., Atlanta, GA (US); Georgia Tech Research Corporation, Atlanta, GA (US)

(72) Inventors: Eric Gaucher, Atlanta, GA (US); Lily Tran, Atlanta, GA (US); M. G. Finn, Atlanta, GA (US); Soumen Das, Atlanta, GA (US)

(21) Appl. No.: 18/701,567

(22) PCT Filed: Oct. 10, 2022
(86) PCT No.: PCT/US2022/077855

§ 371 Date: Apr. 15, 2024

Related U.S. Application Data
(60) Provisional application No. 63/256,271, filed on Oct. 15, 2021.

(51) Int. Cl. A61K 38/44; A61K 9/14; A61K 9/51; A61K 38/00; C12N 9/06

(52) U.S. Cl. CPC A61K 38/44; A61K 9/14; A61K 9/5184; C12N 9/0048; C12Y 107/03003; A61K 38/00; C12N 2795/18123

(57) ABSTRACT

Systems for enhanced delivery of functional, recombinant soluble uricase enzymes with long serum residence, low immunogenicity and potential for modification have been developed. Compositions and methods of use thereof of nanoparticles including recombinant soluble uricase enzymes are provided for oral administration to a subject. The nanoparticle compositions include recombinant soluble uricase enzymes that are not PEGylated, and have a serum residence time of hours, days or weeks following oral administration. A reduced level of side effects of the formulation provides an effective and safe drug delivery platform for treating Tumor Lysis syndrome and Lesch-Nyhan disease, as well as hyperuricemia and associated diseases or disorders.

### CROSS-REFERENCE TO RELATED APPLICATIONS

This application claims the benefit of and priority to U.S. Ser. No. 63/256,271, filed on Oct. 15, 2021, which is incorporated by reference herein in its entirety.

### STATEMENT REGARDING FEDERALLY SPONSORED RESEARCH OR DEVELOPMENT

This invention was made with government support under grant number 5R01AR069137 awarded by the National Institutes of Health (NIH). The government has certain rights in the invention.

### REFERENCE TO SEQUENCE LISTING

The Sequence Listing submitted as an xml file named “GSURF_2021_016_02_PCT.xml,” created on Sep. 27, 2022, and having a size of 21,860 bytes is hereby incorporated by reference pursuant to 37 C.F.R. § 1.834(c)(1).

### FIELD OF THE INVENTION

The invention is generally directed to compositions and methods for delivery of therapeutic uricase enzymes with enhanced serum residence time and reduced autoimmune activity.

### BACKGROUND OF THE INVENTION

Uric acid is a highly insoluble metabolic product mostly of purine metabolism generated from the breakdown of DNA, RNA and ATP. Many non-human mammals have a functional uricase enzyme to metabolize uric acid into 5-hydroxyisourate (which is then metabolized to allantoin) and have plasma uric acid levels in the 1-2 mg/dL range. Serum uric acid levels are higher in the Lesser and Great Apes (including humans) due to parallel nonsense mutations that caused a pseudogenization of the uricase gene via premature stop codons thought to have occurred during the mid-Miocene (Wu X W et al., J Mol Evol 34, 78-84 (1992)) (˜15 million years ago, Ma). The concentrations of uric acid are higher in human serums (3-7 mg/dL for men and 2-6 mg/dL for women) than in closely related primates that have a functional uricase (0.4-0.8 mg/dL in Old World Monkeys), and hyperuricemia in humans is uric acid in blood concentration greater than 6.8 mg/dL. In 2016, the prevalence rate of hyperuricemia in the US was estimated at 20% (Li L et al., Am J Transl Res; 12(7):3167-3181 (2020)).

Elevated uric acid levels in humans can lead to several diseases, including Gout, hypertension, and Tumor Lysis Syndrome (TLS), as well as Lesch-Nyhan disease. Gout occurs where uric acid crystallizes in the tissues and is the most common inflammatory arthritis related to elevated uric acid levels. Recent reports estimate a gout incidence of 0.58-2.89 per 1,000 persons per year, is more prevalent in men than in women and increases in age. Other illnesses related to gout include erectile dysfunction, atrial fibrillation, obstructive sleep apnea, osteoporosis, and venous thromboembolism. Hyperuricemia occurs in 25-50% of hypertensive patients, and in up to 90% of those with new onset hypertension. Some studies suggest that high levels of uric acid confer a risk equivalent to the risk cholesterol poses for cardiovascular events. Hyperuricemia is also a reported risk factor for the progression of renal disease; over 95% of patients with gout have interstitial renal disease at autopsy. Most recently, uric acid has been shown to directly cause acute inflammation by stimulating the production of IL-1β in the inflammasome. Uric acid has also been recognized as a predictive marker for cardiovascular disease, hypertension, and renal disease. Tumor Lysis Syndrome (TLS) occurs when a large number of cancer cells die within a short period and release their contents into the blood. Generally, 1-5 days after chemotherapy starts, tumor lysis may occur, resulting in accumulation of uric acid crystals in the kidneys due to the release of massive amounts of purine DNA from lysed cells during cancer treatments. TLS can lead to acute kidney injury, fatal arrhythmia, and death.

Several drugs have been marketed for the treatment of hyperuricemia, TLS and Lesch-Nyhan disease. Allopurinol is a small molecule xanthine oxidoreductase inhibitor and helps regulate the production of uric acid but fails to reduce urate concentration (a salt or ester of uric acid) to safe levels for almost half of the people suffering with gout. An endogenous or recombinantly-expressed uricase from the yeast Aspergillus flavus was the first therapeutic uricase available in the clinic (marketed as Uricozyme and Rasburicase/Elitek, respectively). However, these enzymes are highly antigenic and repeated administration results in allergic reactions, anaphylaxis, and sometimes death due to antibody development against uricase epitopes. The FDA-approved uricase therapeutic, Pegloticase (KRYSTEXXA®) is a PEG-modified form of a pig-baboon chimeric uricase. However, in phase III clinical studies, 18% of patients discontinued treatment KRYSTEXXA® in response to serious adverse events, and less than 50% of patients met defined endpoints in lowering blood serum uric acid levels and resolution of gout. KRYSTEXXA® is PEGylated because the non-PEGylated form has a circulation half-life of minutes. In general, uricase enzymes are unstable unless PEGylated, however the PEGylated versions of these enzymes are known to elicit strong immune responses in human patients and often result in allergic reactions, anaphylaxis, and in some cases, death due to antibody development against uricase epitopes.

Thus, there remains a lack of effective methods for delivering uricases to subjects in need thereof. There also remains a need to develop more robust formulations and methods for delivering therapeutics for hyperuricemia.

Therefore, it is an object of the present invention to provide formulations for the enhanced treatment of hyperuricemia, Tumor Lysis syndrome and Lesch-Nyhan disease with minimal autoimmunity or systemic toxicity.

It is another object of the present invention to provide formulations and methods for delivering uricase enzymes that are safe and effective for use in humans.

### SUMMARY OF THE INVENTION

Systems for enhanced delivery of recombinant soluble uricase enzymes, with long serum residence and/or low immunogenicity have been developed. Compositions and methods of use thereof of nanoparticles including recombinant functional uricase enzymes are provided for administration to a subject in need thereof, such as a human with hyperuricemia. The recombinant functional uricase enzymes are not PEGylated, and have a serum residence time of hours, days, or weeks following administration.

Nanoparticles including a plurality of functional recombinant uricase enzyme molecules each independently having an amino acid sequence of any one or more of SEQ ID NOs:1-8 are described. The nanoparticles generally have a diameter of between about 50 nm and 1,000 nm, inclusive, as determined by Dynamic Light Scattering (DLS) analysis. For example, in some forms, the diameter of the nanoparticle is between about 75 nm and about 150 nm, as measured by DLS analysis. In particular forms, the diameter of the nanoparticle is about 100 nm, as measured by DLS analysis.

In some forms, the uricase nanoparticle is formed from only a plurality of functional recombinant uricase enzyme molecules. For example, in particular forms, the nanostructure is formed by non-covalent interactions between nine or more functional recombinant uricase enzyme molecules each independently having an amino acid sequence of any one or more of SEQ ID NOs:1-8. In other forms, the uricase nanoparticle is a nanostructure formed only of (a) a plurality of functional recombinant uricase enzyme molecules; and (b) one or more cross-linker molecules. For example, in some forms the nanostructure is an assembly of nine or more functional recombinant uricase enzyme molecules and one or more cross-linker molecules that cross-link two or more of the functional recombinant uricase enzyme molecules within the nanostructure. In preferred forms, the uricase nanoparticles are formed by self-assembly of the plurality of functional recombinant uricase enzyme molecules.

In some forms, the nanoparticle is a virus-like-particle (VLP), including (a) a plurality of functional recombinant uricase enzyme molecules; and (b) viral capsid proteins, whereby the functional recombinant uricase enzyme molecules are encapsulated within the viral capsid proteins.

Exemplary viral capsid proteins are from a virus selected from bacteriophage Q-beta, bacteriophage PP7, and bacteriophage MS. Typically, the viral capsid proteins encapsulate between one and one hundred functional recombinant uricase enzyme molecules. For example, in some forms, the viral capsid proteins encapsulate between ten and thirty functional recombinant uricase enzyme molecules. In a particular form, the viral capsid proteins encapsulate twenty-five functional recombinant uricase enzyme molecules.

In some forms, the VLP is modified by attachment of one or more moieties to an external face of one or more of the viral capsid proteins. Exemplary moieties include a protein, a nucleic acid, a carbohydrate, a polymer, a lipid, a small molecule, a secondary nanoparticle, a microparticle, a cell, and a virus. In some forms, one or more of the moieties are functional moieties, for example, including one or more selected from a targeting moiety, a fluorescent label, a therapeutic agent and a cross-linking agent. In some forms, the nanoparticle encapsulates one or more additional active agents. Exemplary agents include one or more selected therapeutic agents, diagnostic agents, nutraceutical agents, and enzyme catalysts.

Pharmaceutical formulations of nanoparticles including a plurality of functional recombinant uricase enzyme molecules each independently having an amino acid sequence of any one or more of SEQ ID NOs:1-8 are also described. Typically, the formulation is in a form selected from a solution, a dry powder, a tablet, micelles, colloids, nanodroplets, nano-structured hydrogel, nanocrystals, and a nanosuspension.

Methods of treating, retarding development of, or preventing development of hyperuricemia in a subject are also described. Generally, the methods include administering to a subject in need thereof an effective amount of a pharmaceutical formulation of nanoparticles including a plurality of functional recombinant uricase enzyme molecules each independently having an amino acid sequence of any one or more of SEQ ID NOs:1-8 to treat, retard development of, or prevent development of hyperuricemia in the subject. For example, in some forms, the method treats, retards development of, or prevents development of one or more diseases or disorders associated with hyperuricemia in the subject selected from Gout, hypertension, renal disease, Tumor Lysis Syndrome (TLS), and Lesch-Nyhan disease. In some forms, the methods administer the pharmaceutical formulation to the subject via a route selected from oral administration, intramuscular injection, intravenous injection, sub-cutaneous injection, and intra-articular injection. In preferred forms, the methods administer the formulation an amount effective to reduce uric acid in the blood of the subject to an amount less than or equal to 6 mg/dL uric acid in the blood.

Compositions including a gene editing system and a donor sequence encoding one or more uricase enzymes are also provided. Typically, the gene editing system is a CRISPR/Cas system. In some forms, the CRISPR/Cas system includes (a) a single-guide RNA (sgRNA) that targets the human AAVS1 locus and (b) a Cas nuclease or nickase enzyme. In preferred forms, the one or more uricase enzymes encoded by the gene editing system and donor sequence is an ancestral form of a human uricase, or a derivative or variant thereof. In some forms, the one or more uricase enzymes encoded by the gene editing system and donor sequence are functional recombinant uricase enzymes each independently having an amino acid sequence of any one or more of SEQ ID NOs:1-8.

Methods for inducing or increasing the expression or production of uricase enzyme in a subject in need thereof are also described. Typically, the methods include administering to the subject an effective amount of a gene editing system and a donor sequence encoding one or more uricase enzymes are also provided. An exemplary subject has or is at risk of developing hyperuricemia. Therefore, in some forms the methods induce or increase the expression or production of uricase enzyme in an amount effective to treat, retard development of, or prevent development of one or more diseases or disorders associated with hyperuricemia in the subject. Exemplary diseases or disorders include Gout, hypertension, renal disease, Tumor Lysis Syndrome (TLS), and Lesch-Nyhan disease. In some forms, the methods administer to the subject a gene editing system and a donor sequence encoding one or more uricase enzymes in an amount effective to reduce uric acid in the blood of the subject to an amount less than or equal to 6 mg/dL uric acid in blood.

Methods of making uricase nanoparticles are also provided.

Also disclosed are uricases that comprise at least one uricase subunit having an amino acid sequence that is at least about 50%, 55%, 60%, 65% 70%, 75%, 80%, 85%, 90%, 95%, 96%, 97%, 98%, 99% or 100% identical to one or more of the respective amino acid sequences of SEQ ID NOs:1-8. In some forms, the uricase is a functional recombinant uricase enzyme molecule. In some forms, the at least one uricase subunit has a cysteine at least at one or more of amino acid residues selected from position 56,position 236, position 68, position 290, position 223, position 130, position 237, position 60, position 13, position 299, position 25, and position 287.

In some forms, the at least one uricase subunit has a cysteine at least at one or more of amino acid residues selected from position 56, position 236, position 68, position 290, position 223, position 130, position 237, position 60, position 13, position 299,position 25, and position 287, and has improved thermostability compared to a uricase without a cysteine at the corresponding position(s).

In some forms, the at least one uricase subunit has the amino acid sequence of SEQ ID NO:1 and one or more mutations selected from D56C, Q236C, T68C, G290C, A223C, A130C, K237C, G60C, E13C, K299C, M25C, and N287C.

In some forms, the at least one uricase subunit has the amino acid sequence of SEQ ID NO:1 and one or more mutations selected from M25C and N287C. In some forms, the at least one uricase subunit has the amino acid sequence of SEQ ID NO:8. In some forms, the uricase is a homo-tetramer or a hetero-tetramer.

Also disclosed are pharmaceutical compositions comprising a uricase as disclosed.

### BRIEF DESCRIPTION OF THE DRAWINGS

FIGS. 1A-1B depict CRISPR/Cas9-mediated knock-in of ancestral uricase DNA. FIG. 1A is a schematic for CRISPR/Cas9-mediated knock-in of ancestral uricase DNA into human AAVS1 locus in human HEK293 cells using two different single-guide-RNAs. In the AAVS1-RFP_Anc UOX donor vector the genes are flanked by AAVS1 homology arms. Cells transfected with pAAVS1_RFP_Anc UOX plasmid and sgAAVS1-1 (or -2)/Cas9/GFP/E4orf4 expression plasmid were FACS sorted. T2A is a self-cleaving peptide domain that liberates RFP and Uricase from the polypeptide. FIG. 1B is a representative western blot of lysates from HEK293 control and CRISPR+. Samples analyzed by SDS-PAGE and immunoblotted with anti-uricase, anti-catalase and anti-actin antibodies.

FIGS. 2A-2D are line graphs showing intracellular uricase enzymatic activity in control versus CRISPR+ cells (two guide-RNAs, g1 and g2, were each tested on two separate groups of cells to generate four test conditions). All four test conditions of CRISPR+ cells showed decreasing OD293 as an indication of uricase-dependent oxidation of the uric acid substrate. Four concentrations including 100, 200, 400 and 600 μM were assayed.

FIG. 3 is a graph of the size of uricase nanoparticles analyzed by Dynamic Light Scattering (DLS), showing % Intensity (0-90) over Radius (1-1,000 nm).

FIGS. 4A-4C depict oral gavage treatment of Qβ-AncUOX vesicles in mice. FIG. 4A is a cartoon depicting administering ancestral An67 uricase (AncUOX) encapsulated in a vesicle of Qβ capsid protein, termed Qβ-AncUOX, to Uricase knockout mice via oral gavage. FIG. 4B is a line graph of the ratio of urine uric acid:creatinine (0-12) over age in (4-8 weeks), showing mean±SEM for each of Qβ-AncUOX (solid line) and Qβ-empty (dashed line), respectively. n=4 (two females and two males). WT mice have a ratio≤1.0. FIG. 4C is a bar graph showing the difference in the ratio of urine uric acid to creatinine between Qβ-AncUOX (black) and Qβ-empty (gray), for each of two time points, 4 weeks old and 8 weeks old, respectively. Data are presented as mean±SEM, n=4 in each group (two females and two males). Significance was evaluated using a student t-test in SPSS Statistics.

FIGS. 5A-5B are line graphs showing fraction velocity (0-100%) of an ancestral uricase (An96) over a period of 21 minutes at 30° C., 37° C., and 40° C. (FIG.5A), and of an engineered An96 uricase containing mutations M25C and N287C over a period of 72 hours at 30° C., 37° C., and 40° C. (FIG.5B).

### DETAILED DESCRIPTION OF THE INVENTION

### I. Definitions

The term “nanoparticle,” as used herein, generally refers to a structure of any shape having a diameter from about 1 nm up to, but not including, about 1 micron, more preferably from about 5 nm to about 500 nm, most preferably about 100 nm. Nanoparticles having a spherical shape are generally referred to as “nanospheres.” Non-limiting examples of nanoparticles include virus-like particles, particles formed of high-order structures of proteins, and soft nanoparticles, e.g., micelles, colloids, liposomes, vesicles, nanodroplets nano-structured hydrogel, nanocrystals, and nanosuspension. Soft nanoparticles generally dissolve or dissemble to release agents.

The term “IC₅₀,” as used herein, refers to a concentration of an inhibitor (or tested agent) where the response (or binding) is reduced by half.

The term “polymer” refers to a chemical entity with a plurality of repeating units generally bonded covalently. In some forms, a polymer has a molecular weight greater than 500 or 1,000, or more. Non-limiting exemplary polymers include polyamino acids, naturally occurring, and synthetic chemical compounds.

The term “pharmaceutically acceptable,” as used herein, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration.

The terms “biocompatible” and “biologically compatible,” as used herein, generally refer to materials that are, along with any metabolites or degradation products thereof, generally non-toxic to the recipient, and do not cause any significant adverse effects to the recipient. Typically, biocompatible materials are materials which do not elicit a significant inflammatory or immune response when administered to a patient.

The term “hydrophilic,” as used herein, refers to the property of having affinity for water. For example, hydrophilic polymers (or hydrophilic polymer segments) are polymers (or polymer segments) which are primarily soluble in aqueous solutions and/or have a tendency to absorb water. In general, the more hydrophilic a polymer is, the more that polymer tends to dissolve in, mix with, or be wetted by water.

The term “hydrophobic,” as used herein, refers to the property of lacking affinity for or repelling water. For example, the more hydrophobic a polymer (or polymer segment), the more that polymer (or polymer segment) tends to not dissolve in, not mix with, or not be wetted by water.

The term “therapeutic agent” refers to an agent that can be administered to prevent or treat a disease or disorder. Therapeutic agents can be a nucleic acid, a nucleic acid analog, a small molecule, a peptidomimetic, a protein, peptide, carbohydrate or sugar, lipid, or surfactant, or a combination thereof.

The terms “treating” and “retarding development of” a disease, disorder, or condition occurring in an animal which has or may be predisposed to the disease, disorder and/or condition mean inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. In some forms, the animal has been diagnosed with the disease or disorder. In other forms, the animal has not yet been diagnosed as having the disease or disorder. The terms “preventing” and “preventing development of” used in the context of a disease, disorder, or condition in an animal mean inhibiting the initiation or development of the disease, disorder or condition, e.g., stopping the animal from developing the disease, disorder or condition, or impeding its progress; and/or preventing the advancement or continuation of at least one symptom of the particular disease or condition, even if the underlying pathophysiology is not affected, such as preventing pain in a subject by administration of an analgesic agent, even though such agent does not treat the cause of the pain.

The term “targeting moiety” as used herein refers to a moiety that localizes to or away from a specific locale. The moiety may be, for example, a protein, nucleic acid, nucleic acid analog, carbohydrate, or small molecule. The entity may be, for example, a therapeutic compound such as a small molecule, or a diagnostic entity such as a detectable label. The locale may be a tissue, a particular cell type, or a subcellular compartment.

The term “prolonged residence time” means an increase in the time required for an agent to be cleared from a patient's body, or organ or tissue of that patient. In certain forms, “prolonged residence time” refers to an agent that is cleared with a half-life that is 10%, 20%, 50% or 75% longer than a standard of comparison such as a comparable agent without association with or encapsulation within a delivery vehicle such as a nanoparticle. In some forms, “prolonged residence time” refers to an agent that is cleared with a half-life of 2, 5, 10, 20, 50, 100, 200, 500, 1000, 2000, 5000, or 10000 times longer than a standard of comparison such as a comparable agent without a nanoparticle that specifically target specific cell types associated with a disease or condition.

The term “therapeutically effective amount” means an amount of the therapeutic agent that, when incorporated into and/or onto particles described herein, produces some desired effect at a reasonable benefit/risk ratio applicable to any treatment. The effective amount may vary depending on such factors as the disease or condition being treated, the particular formulation being administered, the size of the subject, or the severity of the disease or condition.

The terms “incorporated” and “encapsulated” means incorporating, formulating, or otherwise including an agent into and/or onto a composition, regardless of the manner by which the agent or other material is incorporated.

The term “fragment” means a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, 100, 200, 300, 400, 500, 600, 700, 800, 900, or 1000 nucleotides or amino acids.

The terms “isolated,” “purified,” or “biologically pure” mean material that is free to varying degrees from components which normally accompany it as found in its native state. “Isolate” denotes a degree of separation from original source or surroundings. “Purify” denotes a degree of separation that is higher than isolation.

By “protein” or “polypeptide” or “peptide” is meant any chain of more than two natural or unnatural amino acids, regardless of post-translational modification (e.g., glycosylation or phosphorylation), constituting all or part of a naturally occurring or non-naturally occurring polypeptide or peptide, as is described herein.

By “subject” is meant a mammal, including, but not limited to, a human or non-human mammal, such as a bovine, equine, canine, ovine, or feline. The subject is preferably a mammal in need of treatment, e.g., a subject that has been diagnosed with a disease or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred form, the mammal is a human.

### II. Compositions

Compositions of nanoparticles including recombinant, functional, soluble uricase enzymes are provided for administration to a subject. The recombinant, functional, soluble uricase enzymes are not PEGylated, and have a serum residence time of hours, days or weeks following administration in vivo. Preferably, the recombinant, functional, soluble uricase enzymes are engineered to include ancestral genomic uricase-like amino acid sequences. Exemplary recombinant, functional, soluble uricase enzymes include polypeptides having an amino acid sequence including any one or more or SEQ ID NOs:1-8. In some forms, the nanoparticles are tetrameric structures of a multiplicity of recombinant, functional, soluble uricase enzymes, optionally cross-linked for enhanced stability. In other forms, the nanoparticles include recombinant, functional, soluble uricase enzymes encapsulated within, or associated with virus-like particles. Exemplary virus like particles include Leviviridae-derived capsids.

In some forms, the nanoparticles are sized for uptake within the gut following oral administration. An exemplary way to measure the diameter of a nanoparticle is by using Dynamic light Scattering (DLS). The nanoparticle may have a diameter of between about 1,000 nm and about 10 microns, inclusive, as measured by DLS, for example, between about 100 nm and about 1 micron, inclusive, between about 10 nm and about 500 nm, inclusive, between about 20 nm and about 500 nm, inclusive, or between about 25 nm and about 250 nm, inclusive. For example, the nanoparticle can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 60 nm to 400 nm, from 50 nm to 350 nm, from 50 nm to 300 nm, or from 50 nm to 200 nm. In preferred forms the nanoparticles can have a diameter less than 400 nm, less than 300 nm, or less than 200 nm. The preferred range is between 50 nm and 300 nm, or 25 nm and 250 nm, or 80 nm and 150 nm, inclusive. In particular forms a nanoparticle has a diameter of about 100 nm.

The particle or nanoparticle can have a zeta potential between −100 mV and +100 mV, inclusive, between −50 mV and +50 mV, inclusive, between −40 mV and +40 mV, inclusive, between −30 mV and +30 mV, inclusive, between −20 mV and +20 mV, inclusive, between −10 mV and +10 mV, inclusive, or between −5 mV and +5 mV, inclusive. The particle or nanoparticle can have a negative zeta potential. The particle can have a positive zeta potential. In some forms the particle has a substantially neutral zeta potential, i.e., the zeta potential is approximately 0 mV. In some forms, the particle has a zeta potential of approximately between −20 mV and +20 mV, inclusive, more preferably between −10 mV and +10 mV, inclusive. In some forms, the zeta potential is between 0 and +100, inclusive, e.g., between 0 mV and +40 mV, inclusive.

### A. Uricase Enzymes

Nanoparticles formed of a multiplicity of molecules of recombinant, functional, soluble uricase enzymes are described. Uricase (Urate oxidase) is present in liver, where it forms a large electron-dense paracrystalline core in many peroxisomes. Uricase is a tetramer of identical subunits, each including a possible type 2 copper-binding site. Urate oxidase is a homo-tetrameric enzyme containing four identical active sites situated at the interfaces between its four subunits. Sequence analysis of several organisms has determined that there are 24 amino acids which are conserved, and of these, 15 are involved with the active site.

Active, functional uricase enzymes are not present in humans, however non-functional ancestral uricases-like components are present within the human genome. Preferred uricases are functional ancestral forms of mammalian uricases, for example, engineered to be functional variants of the non-functional ancestral human enzymes. In a preferred form, the uricase enzyme is functional and minimally immunogenic in humans. In a most preferred form, the enzyme is functional and non-immunogenic in humans.

In particular forms, a non-functional human ancestral uricase that includes two premature stop codons is used as a basis for engineering a functional uricase that is non-immunogenic in humans in the absence of PEG. In some forms, the uricase represents an ancestral form of a human uricase or a variant (e.g., mutated form) thereof. Useful ancestral-based uricase enzymes are described in U.S. Pat. Nos. 8,940,861 and 8,586,535, the contents of which are hereby incorporated by reference in their entirety. Exemplary amino acid sequences for an ancestral uricases are set forth below:

(SEQ ID NO: 1)
MAHYHNDYKKNDEVEFVRTGYGKDMVKVLHIQRDGKYHSIKEVATSVQLT
LSSKKDYLHGDNSDIIPTDTIKNTVHVLAKFKGIKSIEAFAMNICEHFLS
SFNHVIRAQVYVEEVPWKRFEKNGVKHVHAFIHTPTGTHFCEVEQMRSGP
PVIHSGIKDLKVLKTTQSGFEGFIKDQFTTLPEVKDRCFATQVYCKWRYH
QGRDVDFEATWDTVRDIVLEKFAGPYDKGEYSPSVQKTLYDIQVLSLSRV
PEIEDMEISLPNIHYFNIDMSKMGLINKEEVLLPLDNPYGKITGTVKRKL
SSRL
(SEQ ID NO: 2)
MAHYHNDYKKNDEVEFVRTGYGKDMVKVLHIQRDGKYHSIKEVATSVQLT
LASKKDYLKGDNSDIIPTDTIKNTVHVLAKFKGIKSIEAFAMNICEHFLS
SFNHVIRAQVYVEEVPWKRFEKNGVKHVHAFIHTPTGTHRCEVEQMRSGP
PVIHSGIKDLKVLKTTQSGFEGFIKDQFTTLPEVKDRVFATQVYCKWRYH
QGRDVDFEATWDTVRDIVLEKFAGPYDKGEYSPSVQKTLYDIQVLSLSRV
PEIEDMEISLPNIHYFNIDMSKMGLINKEEVLLPLDNPYGKITGTVKRKL
SSRL
(SEQ ID NO: 3)
MAHYHNDYKKNDEVEFVRTGYGKDMVKVLHIQRDGKYHSIKEVATSVQLT
LSSKKCYLHGDNSDIIPTDTIKNTVHVLAKFKGIKSIEAFAMNICEHFLS
SFNHVIRAQVYVEEVPWKRFEKNGVKHVHAFIHTPTGTHFCEVEQMRSGP
PVIHSGIKDLKVLKTTQSGFEGFIKDQFTTLPEVKDRCFATQVYCKWRYH
QGRDVDFEATWDTVRDIVLEKFAGPYDKGEYSPSVCKTLYDIQVLSLSRV
PEIEDMEISLPNIHYFNIDMSKMGLINKEEVLLPLDNPYGKITGTVKRKL
SSRL
(SEQ ID NO: 4)
MAHYHNDYKKNDEVEFVRTGYGKDMVKVLHIQRDGKYHSIKEVATSVQLT
LSSKKDYLHGDNSDIIPCDTIKNTVHVLAKFKGIKSIEAFAMNICEHFLS
SFNHVIRAQVYVEEVPWKRFEKNGVKHVHAFIHTPTGTHFCEVEQMRSGP
PVIHSGIKDLKVLKTTQSGFEGFIKDQFTTLPEVKDRCFATQVYCKWRYH
QGRDVDFEATWDTVRDIVLEKFAGPYDKGEYSPSVQKTLYDIQVLSLSRV
PEIEDMEISLPNIHYFNIDMSKMGLINKEEVLLPLDNPYCKITGTVKRKL
SSRL
(SEQ ID NO: 5)
MAHYHNDYKKNDEVEFVRTGYGKDMVKVLHIQRDGKYHSIKEVATSVQLT
LSSKKDYLHGDNSDIIPTDTIKNTVHVLAKFKGIKSIEAFAMNICEHFLS
SFNHVIRAQVYVEEVPWKRFEKNGVKHVHCFIHTPTGTHFCEVEQMRSGP
PVIHSGIKDLKVLKTTQSGFEGFIKDQFTTLPEVKDRCFATQVYCKWRYH
QGRDVDFEATWDTVRDIVLEKFCGPYDKGEYSPSVQKTLYDIQVLSLSRV
PEIEDMEISLPNIHYFNIDMSKMGLINKEEVLLPLDNPYGKITGTVKRKL
SSRL
(SEQ ID NO: 6)
MAHYHNDYKKNDEVEFVRTGYGKDMVKVLHIQRDGKYHSIKEVATSVQLT
LSSKKDYLHCDNSDIIPTDTIKNTVHVLAKFKGIKSIEAFAMNICEHFLS
SFNHVIRAQVYVEEVPWKRFEKNGVKHVHAFIHTPTGTHFCEVEQMRSGP
PVIHSGIKDLKVLKTTQSGFEGFIKDQFTTLPEVKDRCFATQVYCKWRYH
QGRDVDFEATWDTVRDIVLEKFAGPYDKGEYSPSVQCTLYDIQVLSLSRV
PEIEDMEISLPNIHYFNIDMSKMGLINKEEVLLPLDNPYGKITGTVKRKL
SSRL
(SEQ ID NO: 7)
MAHYHNDYKKNDCVEFVRTGYGKDMVKVLHIQRDGKYHSIKEVATSVQLT
LSSKKDYLHGDNSDIIPTDTIKNTVHVLAKFKGIKSIEAFAMNICEHFLS
SFNHVIRAQVYVEEVPWKRFEKNGVKHVHAFIHTPTGTHFCEVEQMRSGP
PVIHSGIKDLKVLKTTQSGFEGFIKDQFTTLPEVKDRCFATQVYCKWRYH
QGRDVDFEATWDTVRDIVLEKFAGPYDKGEYSPSVQKTLYDIQVLSLSRV
PEIEDMEISLPNIHYFNIDMSKMGLINKEEVLLPLDNPYGKITGTVKRCL
SSRL
(SEQ ID NO: 8)
MAHYHNDYKKNDEVEFVRTGYGKDCVKVLHIQRDGKYHSIKEVATSVQLT
LSSKKDYLHGDNSDIIPTDTIKNTVHVLAKFKGIKSIEAFAMNICEHFLS
SFNHVIRAQVYVEEVPWKRFEKNGVKHVHAFIHTPTGTHFCEVEQMRSGP
PVIHSGIKDLKVLKTTQSGFEGFIKDQFTTLPEVKDRCFATQVYCKWRYH
QGRDVDFEATWDTVRDIVLEKFAGPYDKGEYSPSVQKTLYDIQVLSLSRV
PEIEDMEISLPNIHYFNIDMSKMGLINKEEVLLPLDCPYGKITGTVKRKL
SSRL

Exemplary uricases include one or more uricase subunits having an amino acid sequence that is at least about 50%, 55%, 60%, 65% 70%, 75%, 80%, 85%, 90%, 95%, 96%, 97%, 98%, 99% or 100% identical to one or more of the respective amino acid sequences of SEQ ID NOs:1-8. In some forms, uricases include one or more uricase subunits having an amino acid sequence of SEQ ID NO:1, and has one or more mutations selected from D56C, Q236C, T68C, G290C, A223C, A130C, K237C, G60C, E13C, K299C, M25C and N287C. In some forms, uricases include one or more uricase subunits having an amino acid sequence that is at least about 50%, 55%, 60%, 65% 70%, 75%, 80%, 85%, 90%, 95%, 96%, 97%, 98%, 99% or 100% identical to one or more of the respective amino acid sequences of SEQ ID NOs:1-8, and a cysteine at least at one or more of the following amino acid residues: position 56, position 236, position 68, position 290, position 223, position 130, position 237, position 60, position 13, position 299, position 25, and position 287. In preferred forms, the uricase has a cysteine at least at one or more of the following amino acid residues: position 56, position 236, position 68, position 290, position 223, position 130, position 237, position 60, position 13, position 299, position 25, and position 287 and has improved thermostability compared to a uricase without a cysteine at the corresponding position(s).

In a preferred form, the uricase includes one or more uricase subunits having an amino acid sequence of SEQ ID NO:8, or a functional fragment thereof. In some forms, the uricases forms a homo-tetramer or a hetero-tetramer.

For example, uricases can include one or more uricase subunits having an amino acid sequence that is at least about 50%, 55%, 60%, 65% 70%, 75%, 80%, 85%, 90%, 95%, 96%, 97%, 98%, 99% or 100% identical to one or more of the respective amino acid sequences of SEQ ID NO:1, SEQ ID NO:2, SEQ ID NO:3, SEQ ID NO:4, SEQ ID NO:5, SEQ ID NO:6, SEQ ID NO:7, and SEQ ID NO:8.

In some forms, the uricase includes one or more uricase subunits having an amino acid sequence that is at least about 50%, 55%, 60%, 65% 70%, 75%, 80%, 85%, 90%, 95%, 96%, 97%, 98%, 99% or 100% identical to the amino acid sequence of SEQ ID NO:1. In other forms, the uricase includes one or more uricase subunits having an amino acid sequence that is at least 90% identical to the amino acid sequence of SEQ ID NO:1. In other forms, the uricase includes one or more uricase subunits having an amino acid sequence that is at least 95% identical to the amino acid sequence of SEQ ID NO:1; at least 96% identical to the amino acid sequence of SEQ ID NO:2; at least 95% identical to the amino acid sequence of SEQ ID NO:3; at least 94% identical to the amino acid sequence of SEQ ID NO:4; at least 94% identical to the amino acid sequence of SEQ ID NO:5; at least 93% identical to the amino acid sequence of SEQ ID NO:6; at least 91% identical to the amino acid sequence of SEQ ID NO:7; or is at least 90% identical to the amino acid sequence of SEQ ID NO:8.

In other forms, the uricase includes one or more uricase subunits having an amino acid sequence of SEQ ID NO:1, or SEQ ID NO:2, or SEQ ID NO:3, or SEQ ID NO:4, or SEQ ID NO:5, or SEQ ID NO:6, or SEQ ID NO:7 or SEQ ID NO:8, and where the amino acid at one or more of positions 96, 103, 146, 147, 192, 208, 220, 230 and 303 is lysine (K; Lys). In some forms, the stability of the uricase is enhanced by the presence of an arginine (R; Arg) at amino acid position 175, a Glutamine (Glu; E) at amino acid position 177, a tyrosine (Tyr; Y) at amino acid position 178, a Leucine (L; Leu) at amino acid position 265, a glycine (Gly; G) at amino acid position 273 and/or a serine (Ser; S) or a cysteine (Cys; C) at amino acid position 286 of the amino acid sequence of SEQ ID NO:1, SEQ ID NO:2, SEQ ID NO:3, SEQ ID NO:4, SEQ ID NO:5, SEQ ID NO:6, SEQ ID NO:7 or SEQ ID NO:8. In some forms, the uricase includes one or more uricase subunits having an amino acid sequence that is 100% identical to the amino acid sequence of SEQ ID NO:1; 100% identical to the amino acid sequence of SEQ ID NO:2; 100% identical to the amino acid sequence of SEQ ID NO:3; 100% identical to the amino acid sequence of SEQ ID NO:4; 100% identical to the amino acid sequence of SEQ ID NO:5; 100% identical to the amino acid sequence of SEQ ID NO:6; 100% identical to the amino acid sequence of SEQ ID NO:7 or 100% identical to the amino acid sequence of SEQ ID NO:8.

### 1. CRISPR/Cas-Uricase System

In some forms, the composition includes a genome editing system for introducing a functional uricase gene into a cell, a tissue, an organ, or an organism. In preferred forms, the genome editing system is the CRISPR/Cas system. In some forms, the uricase gene encodes an ancestral-based uricase enzyme such as those described in U.S. Pat. Nos. 8,940,861 and 8,586,535.

CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By transfecting a cell with the required elements including a cas gene and specifically designed CRISPRs, the organism's genome can be cut and modified at any desired location. Methods of preparing compositions for use in genome editing using the CRISPR/Cas systems are described in detail in WO 2013/176772 and WO 2014/018423, which are specifically incorporated by reference herein in their entireties.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more tracr mate sequences operably linked to a guide sequence (e.g., direct repeat-spacer-direct repeat) can also be referred to as pre-crRNA (pre-CRISPR RNA) before processing or crRNA after processing by a nuclease.

In some forms, a tracrRNA and crRNA are linked and form a chimeric crRNA-tracrRNA hybrid where a mature crRNA is fused to a partial tracrRNA via a synthetic stem loop to mimic the natural crRNA: tracrRNA duplex as described in Cong, Science, 15:339(6121):819-823 (2013) and Jinek, et al., Science, 337(6096):816-21 (2012)). A single fused crRNA-tracrRNA construct can also be referred to as a guide RNA or gRNA (or single-guide RNA (sgRNA)). Within an sgRNA, the crRNA portion can be identified as the “target sequence” and the tracrRNA is often referred to as the “scaffold.”

In general, a CRISPR system is characterized by elements that promote the formation of a CRISPR complex at the site of a target sequence. In the context of formation of a CRISPR complex, “target sequence” refers to a sequence to which a guide sequence is designed to have complementarity, where hybridization between a target sequence and a guide sequence promotes the formation of a CRISPR complex. A target sequence can be any polynucleotide, such as DNA or RNA polynucleotides. In some forms, a target sequence is located in the nucleus or cytoplasm of a cell.

There are many resources available for helping practitioners determine suitable target sites once a desired DNA target sequence is identified. For example, numerous public resources, including a bioinformatically generated list of about 190,000 potential sgRNAs, targeting more than 40% of human exons, are available to aid practitioners in selecting target sites and designing the associate sgRNA to affect a nick or double strand break at the site. See also, crispr.u-psud.fr/, a tool designed to help scientists find CRISPR targeting sites in a wide range of species and generate the appropriate crRNA sequences. In one form, the target site is human AAVS1 locus.

In some forms, one or more vectors driving expression of one or more elements of a CRISPR system are introduced into a target cell such that expression of the elements of the CRISPR system direct formation of a CRISPR complex at one or more target sites. While the specifics can be varied in different engineered CRISPR systems, the overall methodology is similar. A practitioner interested in using CRISPR technology to target a DNA sequence (such as human AAVS1 locus) can insert a short DNA fragment containing the target sequence into a guide RNA expression plasmid. The sgRNA expression plasmid contains the target sequence (about 20 nucleotides), a form of the tracrRNA sequence (the scaffold) as well as a suitable promoter and necessary elements for proper processing in eukaryotic cells. Such vectors are commercially available (see, for example, Addgene). Many of the systems rely on custom, complementary oligos that are annealed to form a double stranded DNA and then cloned into the sgRNA expression plasmid. Co-expression of the sgRNA and the appropriate Cas enzyme from the same or separate plasmids in transfected cells results in a single or double strand break (depending on the activity of the Cas enzyme) at the desired target site.

In some embodiments, a vector includes a regulatory element operably linked to an enzyme-coding sequence encoding a CRISPR enzyme, such as a Cas protein.

Non-limiting examples of Cas proteins include Casl, CaslB, Cas2, Cas3, Cas4, Cas5, Cas6, Cas7, Cas8, Cas9 (also known as Csnl and Csxl2), CaslO, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, CsxlO, Csxl6, CsaX, Csx3, Csxl, Csxl5, Csfl, Csf2, Csf3, Csf4, homologues thereof, or modified versions thereof. In some forms, the unmodified CRISPR enzyme has DNA cleavage activity, such as Cas9. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some forms, an enzyme coding sequence encoding a CRISPR enzyme is codon optimized for expression in particular cells, such as liver cells and kidney cells.

In some embodiments, one or more of the elements of CRISPR system are under the control of an inducible promoter, which can include inducible Cas, such as Cas9.

In some forms, the genome editing composition includes a donor polynucleotide to induce gene correction, gene replacement, gene tagging, transgene insertion, nucleotide deletion, gene disruption, gene mutation, etc. In some forms, the donor polynucleotide includes nucleic acid encoding a functional uricase, optionally nucleic acid encoding a reporter protein. In one particular form, the composition includes CRISPR/Cas genome editing and a donor polynucleotide includes nucleic acid encoding red fluorescent protein and ancestral uricase An67.

Exemplary, non-limiting sgRNA sequences can target human AAVS1 locus include:

(SEQ ID NO: 9)
gRNA1: 5′ACCCCACAGTGGGGCCACT3′
(SEQ ID NO: 10)
gRNA2: 5′CTAGGGACAGGATTGGTGAC3′

### 2. Uricase Nanostructures

In some forms, a multiplicity of molecules of the uricase enzyme are combined to form higher-order structures having nanometer dimensions. For example, in some forms, nanostructures include a multiplicity of recombinant, functional uricase enzymes, each independently having an amino acid sequence of any one or more of SEQ ID NOs:1-8. In some forms, the nanostructures include between nine and one hundred recombinant, functional uricase enzyme molecules, each independently having an amino acid sequence of any one or more of SEQ ID NOs:1-8

In some forms, the nanoparticles include uricase enzyme molecules non-covalently bound to one another. For example, in some forms, nanoparticles are formed from the self-assembly of a multiplicity of uricase enzymes. Therefore, in some forms the nanoparticles contain only self-assembled uricase enzyme molecules, as well as solvent and/or co-factor molecules. In other forms, the nanoparticles are formed of self-assembled uricase enzyme molecules that are cross-linked together, for example, to enhance the stability of the nanoparticles. Therefore, in some forms the nanoparticles include uricase enzyme molecules covalently bound to one another. In other forms, the nanoparticles include uricase enzyme molecules covalently and/or non-covalently bound to one another.

### B. Virus-Like Particles (VLPs)

In some forms, the nanoparticles of recombinant, functional, soluble uricase enzyme are virus-like particles (VLPs). For example, in some forms, the nanoparticles include recombinant, functional, soluble uricase enzyme encapsulated within, or otherwise associated with a viral capsid, or viral capsid proteins. In a preferred form, a multiplicity of recombinant soluble uricase enzyme molecules is encapsulated within a viral capsid. For example, in some forms, VLPs include a multiplicity of recombinant, functional uricase enzymes, each independently having an amino acid sequence of any one or more of SEQ ID NOs:1-8 encapsulated within and/or associated with viral capsid proteins.

The single-strand RNA bacteriophages offer certain advantages for such applications. VLPs can be produced in large quantities by self-assembly of a single coat protein polypeptide expressed from a plasmid, thus allowing extensive genetic manipulation of the capsid without the constraints imposed by the necessity to maintain virus viability. Engineering is further facilitated by detailed knowledge of the three-dimensional structures of RNA phages. Therefore, in various forms, uricase nanoparticles include VLPs formed from viral capsid proteins of single-stranded RNA bacteriophages.

In some forms, the VLPs include up to 5, or 10, or 15, or 20, or 25, or 30, or 35, or 40, or 45, or more than 50 copies of recombinant soluble uricase enzymes. For example, in some forms, the VLPs include between one and one hundred recombinant, functional uricase enzyme molecules, each independently having an amino acid sequence of any one or more of SEQ ID NOs:1-8 encapsulated within and/or associated with viral capsid proteins. It has been established that enzymes entrained within VLPs are significantly stabilized toward thermal and chaotropic denaturation, leading to enhanced, long-lived catalytic activity when the enzyme substrates and products can diffuse into and out of the capsid shell, such as uric acid. Therefore, in some forms, recombinant soluble uricase enzymes packaged into viral-like particles have enhanced, and prolonged catalytic activity as compared with an equivalent amount of the recombinant soluble uricase enzymes alone.

### 1. Viral Capsid Proteins

Preferred virus capsids are stable toward thermal denaturation at temperatures up to 80-100° C., chaotropic agents, and to extremes of pH. Exemplary viral-like particles that are stable toward thermal denaturation at temperatures up to 80-100° C., chaotropic agents, and to extremes of pH include Leviviridae-derived capsids. Therefore, in preferred forms, the uricase nanoparticles include VLPs formed from viral capsid proteins of Leviviridae viruses. Exemplary Leviviridae-derived capsids include as Qβ, PP7, and MS2 virus capsids.

a. Bacteriophage Q-beta VLPs

In some forms, compositions include recombinant soluble uricase enzymes packaged into viral-like particles (VLPs), or vesicles, composed of the bacteriophage Q-beta (Qβ) capsid protein.

Bacteriophage Qbeta (Qubevirus durum), referred to as Qbeta or Qβ, is a positive-strand RNA virus which infects bacteria that have F-pili, most commonly Escherichia coli. The Qβ capsid proteins self-assemble to form an icosahedral viral capsid with a T=3 symmetry, having a diameter of about 26 nm, and including 89 capsid proteins dimers, 178 capsid proteins in total. The capsid protein is involved in viral genome encapsidation through the interaction between a capsid protein dimer and the multiple packaging signals present in the RNA genome. Binding of the capsid proteins to the viral RNA induces a conformational change required for efficient T=3 shell formation. Bacteriophage Qβ coat protein forms uniform virus-like particles when expressed recombinantly in a variety of organisms.

b. Bacteriophage PP7 VLPs

In some forms, compositions include recombinant soluble uricase enzymes packaged into viral-like particles (VLPs), or vesicles, composed of the bacteriophage PP7 capsid protein. The RNA phage PP7 infects Pseudomonas aeruginosa bacteria. The phage PP7 particle includes a plus stranded genomic RNA (3588 nucleotides), surrounded by 180 copies of the coat protein, each 127 amino acid residues long (Olsthoorn et al., Virology 206, 611-625 (1995); Tars, et al., Virology 272, 331-337 (2000)). In addition, the particles of PP7 and all related phages contain a single copy of a maturation protein. This protein is responsible for attachment of the phage particles to the bacterial pili, which is the first step in the infection.

The stability of a virus-like particle (VLP) is an important consideration for its use in nanobiotechnology. The icosahedral capsid of the RNA bacteriophage PP7 is cross-linked by disulfide bonds between coat protein dimers at its 5-fold and quasi-6-fold symmetry axes, providing enhanced stability to VLPs formed from PP7 capsid proteins. In some forms, the PP7 capsid is a modified PP7, for example, modified by attachment of a peptide, carbohydrate, small molecule or nucleic acid to the viral capsid. Modified PP7 VLPs are described in Zhao, et al., ACS Nano. 2019 Apr. 23; 13(4):4443-4454, which is incorporated by reference herein in its entirety. Therefore, modified PP7 VLPs capable of encapsulating the uricase enzymes are also described for use as uricase nanoparticles. The structure of the coat protein is a five-stranded β-sheet with two α-helices and a hairpin. When the capsid is assembled, the helices and hairpin face the exterior of the particle, while the β-sheet faces the interior.

c. Bacteriophage MS2 VLPs

In some forms, compositions include recombinant soluble uricase enzymes packaged into viral-like particles (VLPs), or vesicles, composed of the bacteriophage MS2 capsid proteins. Bacteriophage MS2 (Emesvirus zinderi), commonly called MS2, is an icosahedral, positive-sense single-stranded RNA virus that infects the bacterium Escherichia coli and other members of the Enterobacteriaceae. An MS2 virion (viral particle) is about 27 nm in diameter, as determined by electron microscopy, and includes 180 copies of the coat protein (organized as 90 dimers) arranged into an icosahedral shell with triangulation number T=3, protecting the genomic RNA inside. The virion has an isoelectric point (pI) of 3.9.

### 2. Modifications of Viral Capsids and VLPs

In some forms, compositions include recombinant soluble, functional uricase enzymes packaged into viral-like particles (VLPs), or vesicles, that are modified. In some forms, the uricase nanoparticles include VLPs formed with modified viral capsids derived from the Q-beta bacteriophage, the MS2 bacteriophage, or the PP7 bacteriophage. In some forms, uricase VLPs are modified by addition of one or more moieties selected from a protein, a nucleic acid, a carbohydrate, a polymer, a lipid, a small molecule, a nanoparticle, a microparticle, a cell and a virus. For example, in some forms, uricase VLPs are modified by addition of one or more functional moieties. Exemplary functional moieties are selected from a targeting moiety, a fluorescent label, a therapeutic agent, and a cross-linking agent.

The VLPs are highly stable toward chemical manipulation of the capsid protein, giving rise to a variety of modifications that can be used to further stabilize the particles. For example, the multivalent attachment of polymers can render the particles nearly indestructible, as well as shield them from binding to undesired surfaces or proteins. Chemical reactions using small-molecule reagents also result in the modification of packaged enzymes, sometimes abrogating catalytic activity, but the attachment of large, branched molecules takes place exclusively on the VLP surface, keeping the entrained enzymes active. Therefore, in some forms, the VLPs include polymers or other moieties attached to the particles. In some forms nanoparticles include polymers to shield them from binding to undesired surfaces or proteins, enhance solubility, alter zeta potential or other surface charges, reduce antigenicity, increase serum residence, increase half-life of encapsulated enzymes, reduce non-specific binding, or enhance penetration into a specific tissue or organ, or combinations thereof.

The addition of moieties, or molecules (carbohydrates, proteins, nucleic acids, polymers, etc.) to VLPs can impart desired functional and/or structural properties to the package (such as enhanced stability toward stomach fluid, anchoring on the intestinal mucosa, and immunological shielding) while retaining uricase activity. Preliminary results have been generated using the Qβ particle, and provide a template for using other platforms, such as the more robust platform PP7 (71). Preferably, the VLPs are stable at 4° and the enzymatic properties are constant for at least 12 months, 16 months, 18 months, 20 months, 24 months, 48 months, or longer than 48 months when stored at this temperature. Preferably, the VLPs are non-immunogenic or have very low immunogenicity in a mammalian recipient of the nanoparticles.

a. Targeting Moieties

In some forms, uricase viral-like particles (VLPs), or vesicles, are modified by the addition of one or more targeting moieties. For example, the composition can include one or more targeting moieties associated with, linked, conjugated, or otherwise attached directly or indirectly to the VLP, or nanoparticle or other delivery vehicle thereof. The targeting elements may refer to elements that bind to or otherwise localize the nanoparticles to a specific locale. The locale may be a tissue, a particular cell type, or a subcellular compartment. In some forms, the nanoparticles include targeting moieties that specifically bind to targeted molecules. A positive feedback loop is created when the nanoparticles release an inducing agent that causes a targeted cell, tissue or organ to increase the expression or bioavailablity of the molecules specifically recognized by the targeting moiety.

In some forms, the targeting moiety is pH sensitive, lactate sensitive, or acid sensitive. In some forms, the moiety is one that keeps the composition away from the immune cells, or healthy cells.

Representative targeting moieties include, but are not limited to, antibodies and antigen binding fragments thereof, aptamers, peptides, and small molecules. The targeting moiety can be conjugated to a hydrophobic group, e.g., a polymer that incorporates into the nanoparticle. Typically, the targeting moiety is displayed on the outer shell of the nanoparticle. In some forms, the outer shell serves as a shield to prevent the nanoparticles from being recognized by a subject's immune system, thereby increasing the half-life of the nanoparticles in the subject. The nanoparticles are suitable for systemic, intraperitoneal, oral, pulmonary, or topical administration. The nanoparticles also optionally include a detectable label, for example, a fluorophore or NMR contrast agent that allows visualization of nanoparticles within the diseased area.

The targeting moiety can be an antibody or antigen binding fragment thereof. The targeting moieties should have an affinity for a cell-surface receptor or cell-surface antigen on the targeted organs, tissues, cells, and/or subcellular organelles.

The targeting moiety can specifically recognize and bind to a target molecule specific for a cell type, a tissue type, or an organ. The targeted molecule can be a cell surface polypeptide, lipid, or glycolipid. The target molecule can be a receptor that is selectively expressed on a specific cell surface, a tissue or an organ. Cell specific markers can be for specific types of cells including, but not limited to stem cells, skin cells, blood cells, immune cells, muscle cells, nerve cells, cancer cells, virally infected cells, and organ specific cells. The cell markers can be specific for endothelial, ectodermal, or mesenchymal cells. Representative cell specific markers include, but are not limited to, cancer specific markers.

In some forms, the targeting moiety is a positively charged molecule at a physiological pH, and/or at a pH of a targeted region. In preferred forms, the targeting moiety is a positively charged therapeutic agent at physiological pH, and/or at pH of the target site. In some forms, these modulators with targeting properties are used to target a further therapeutic agent to address one or more aspects of the disease e.g., one for treating the cause of the disease, and another for alleviating one or more disease-associated symptoms.

In further forms, therapeutic agents are modified to have desired targeting functionalities. For example, amine groups are added to the therapeutic agents, or their derivatives to make the overall charges of the targeted agents positive.

b. Polymers

In some forms, uricase viral-like particles (VLPs), or vesicles, are modified with polymers. Preferred polymers include biocompatible polymers. In some forms, the biocompatible polymer(s) is biodegradable or bioabsorbable. In other form, the polymer is non-degradable. In other forms, the particles are a mixture of degradable and non-degradable particles.

In some forms, the VLPs include one or more biocompatible polymer(s) including, but not limited to, polyamino acids; cyclodextrin-containing polymers, in particular cationic cyclodextrin-containing polymers, such as those described in U.S. Pat. No. 6,509,323; polymers prepared from lactones such as poly(caprolactone) (PCL); polyhydroxy acids and copolymers thereof such as poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), and blends thereof, polyalkyl cyanoacralate, polyurethanes, poly(valeric acid), and poly-L-glutamic acid; hydroxypropyl methacrylate (HPMA); polyanhydrides; other polyesters; polyorthoesters; poly(ester amides); polyamides; poly(ester ethers); polycarbonates; polyalkylenes such as polyethylene and polypropylene; polyalkylene glycols such as poly(ethylene glycol) (PEG) and polyalkylene oxides (PEO), and block copolymers thereof such as polyoxyalkylene oxide (“PLURONICS®” or block copolymers containing PEG where PEG has a molecular weight of any values within the range of 300 Daltons to 1 MDa); polyalkylene terephthalates such as poly(ethylene terephthalate); ethylene vinyl acetate polymer (EVA); polyvinyl alcohols (PVA); polyvinyl ethers; polyvinyl esters such as poly(vinyl acetate); polyvinyl halides such as poly(vinyl chloride) (PVC), polyvinylpyrrolidone; polysiloxanes; polystyrene (PS); and celluloses including alkyl celluloses, hydroxyalkyl celluloses, cellulose ethers, cellulose esters, nitro celluloses, hydroxypropylcellulose, and carboxymethylcellulose; polymers of acrylic acids including poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), and poly(octadecyl acrylate) (jointly referred to herein as “polyacrylic acids”); polydioxanone and its copolymers; polyhydroxyalkanoates; polypropylene fumarate; polyoxymethylene; poloxamers; poly(butyric acid); trimethylene carbonate; and polyphosphazenes.

Examples of preferred natural polymers include proteins such as albumin, collagen, gelatin and prolamines, for example, zein, and polysaccharides such as alginate.

Copolymers of the above, such as random, block, or graft copolymers, or blends of the polymers listed above can also be used.

Functional groups on the polymer can be capped to alter the properties of the polymer and/or modify (e.g., decrease or increase) the reactivity of the functional group. For example, the carboxyl termini of carboxylic acid contain polymers, such as lactide- and glycolide-containing polymers, may optionally be capped, e.g., by esterification, and the hydroxyl termini may optionally be capped, e.g. by etherification or esterification.

The weight average molecular weight can vary for a given polymer but is generally from about 1000 Daltons to 1,000,000 Daltons, 1000 Daltons to 500,000 Dalton, 1000 Daltons to 250,000 Daltons, 1000 Daltons to 100,000 Daltons, 5,000 Daltons to 100,000 Daltons, 5,000 Daltons to 75,000 Daltons, 5,000 Daltons to 50,000 Daltons, or 5,000 Daltons to 25,000 Daltons.

In some forms, the nanoparticles are modified with one or more surfactants. Examples of surfactants include, but are not limited to, L-α-phosphatidylcholine (PC), 1,2-dipalmitoylphosphatidycholine (DPPC), oleic acid, sorbitan trioleate, sorbitan mono-oleate, sorbitan monolaurate, polyoxyethylene (20) sorbitan monolaurate, polyoxyethylene (20) sorbitan monooleate, natural lecithin, oleyl polyoxyethylene (2) ether, stearyl polyoxyethylene (2) ether, lauryl polyoxyethylene (4) ether, block copolymers of oxyethylene and oxypropylene, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400, cetyl pyridinium chloride, benzalkonium chloride, olive oil, glyceryl monolaurate, corn oil, cotton seed oil, and sunflower seed oil, lecithin, oleic acid, and sorbitan trioleate.

In some forms where polyalkylene glycol (e.g., PEG) is used in a composition of polymers to modify the VLPs, PEG surface density may be controlled by varying the amount of PEG in the polymer composition or by mixing a blend of pegylated polymer component and non-pegylated polymer component. The density of PEG or polyalkylene glycol on the surface of formed particles may be evaluated using several techniques.

In some forms, the VLPs are modified by the addition of one or more polymers to possess a specific ζ-potential. For example, in some forms, the VLPs are modified by the attachment of PEG and/or other polymers to the surface to possess ζ-potential of between about 20 mV and about −20 mV, preferably between about 10 mV and about −10 mV, more preferably between about 2 mV and about −2 mV.

### 3. Encapsulation of Other Agents

In some forms, uricase viral-like particles (VLPs), or vesicles, encapsulate one or more additional active agents. For example, in some forms the uricase VLPs encapsulate between one and one hundred molecules of functional uricase enzymes, together with one or more additional active agents selected from therapeutic agents, diagnostic agents, nutraceutical agents and enzyme catalysts. Exemplary additional agents are selected from a protein, a nucleic acid, a carbohydrate, a polymer, a lipid, a small molecule, a secondary nanoparticle, a microparticle, a cell, and a virus. Therefore, in some forms, uricase VLPs encapsulate additional active agents selected from proteins, nucleic acids, carbohydrates, polymers, lipids, small molecules, secondary nanoparticles, microparticles, cells, and viruses, or combinations thereof. In some forms, uricase VLPs encapsulate additional active agents that are diagnostic and/or labelling agents. Therefore, in some forms, the VLPs encapsulate a multiplicity of recombinant, functional uricase enzymes, each independently having an amino acid sequence of any one or more of SEQ ID NOs:1-8 and one or more diagnostic and/or labelling agents. Exemplary diagnostic and/or labelling agents are selected from a fluorescent label, a radioactive label, an infa-red label, a coloring agent, or combinations thereof. In other forms, the VLPs encapsulate an additional therapeutic agent, and/or an additional neutraceutical agent. Therefore, in some forms, the VLPs encapsulate a multiplicity of recombinant, functional uricase enzymes, each independently having an amino acid sequence of any one or more of SEQ ID NOs:1-8 and an additional therapeutic agent, and/or a neutraceutical agent. In a particular form, the Uricase VLPs encapsulate one or more anti-inflammatory agents in addition to uricase enzymes.

### C. Linking Moieties

In some forms, the nanoparticles include a linking chemical moiety that serves to connect, directly or indirectly, one or more moieties to the nanoparticle. The linking chemical moiety can be any organic, inorganic, or organometallic moiety which is polyvalent, so as to provide more than two points of attachment. The linking chemical moiety can be an organic molecule that contains multiple functional groups, or an organic moiety such as a substituted alkyl, unsubstituted alkyl, substituted alkylene, unsubstituted alkylene, substituted alkenyl, unsubstituted alkenyl, substituted alkynyl, unsubstituted alkynyl, substituted aryl, unsubstituted aryl, substituted cycloalkyl, unsubstituted cycloalkyl, substituted heteroalkyl, unsubstituted heteroalkyl, substituted heteroaryl, unsubstituted heteroaryl, substituted heterocyclyl, unsubstituted heterocyclyl, substituted arylalkyl, or unsubstituted arylalkyl.

The functional groups can be any atom or group of atoms that contains at least one atom that is neither carbon nor hydrogen, with the proviso that the groups must be capable of reacting with a nucleophile or an electrophile. Suitable functional groups include halogens (bromine, chlorine, and iodine); oxygen-containing functional groups such as a hydroxyls, epoxides, carbonyls, aldehydes, ester, carboxyls, and acid chlorides; nitrogen-containing functional groups such as amines and azides; and sulfur-containing groups such as thiols. The functional group may also be a hydrocarbon moiety which contains one or more non-aromatic pi-bonds, such as an alkyne, alkene, or diene. The linking chemical moiety can contain at least two different types of functional groups (e.g., one or more amines and one or more hydroxyls, one or more hydroxyls and one or more carboxyls, or one or more halides and one or more hydroxyls). In such cases, the different functional groups present on the linking chemical moiety can be independently addressed synthetically, permitting the covalent attachment of the rest of the compound and the one or more modulators in controlled stoichiometric ratios.

Following reaction with functional groups on the linking chemical moiety, one or more moieties will be covalently joined to the linking chemical moiety via bonds, and/or to the nanoparticle. The identity of these bonds will be determined by the identity of the functional group on the linking chemical moiety, the reactive loci of the rest of the compound and that of the functional group that attaches the modulator (directly or indirectly) to the linking chemical moiety. Examples of suitable bonds that connect the portions of the compound to the linking chemical moiety include —C(O)NH—, —C(O)NR′—, —NR′C(O)—, —C(O)O—, —OC(O)—, —C(O)OCH₂—, —SO₂NR′—, —CH₂R′—, —O—, —NR′H—, —NR′—, —OCONH—, —NHCOO—, —OCONR′—, —NRCOO—, —NHCONH—, —NR′CONH—, —NHCONR′—, —NR′CONR′—, —CHOH—, —CROH—, unsubstituted alkyl (such as unsubstituted C1-C12 alkyl), substituted alkyl (such as substituted C1-C12 alkyl), wherein R′ is hydrogen, halogen (F, Cl, Br, I), hydroxyl, unsubstituted alkyl (such as unsubstituted C1-C12 alkyl), substituted alkyl (such as substituted C1-C12 alkyl), substituted alkylene (such as substituted C1-C12 alkylene), unsubstituted alkylene (such as unsubstituted C1-C12 alkylene), an aryl group, or a heterocyclic group.

### D. Formulations

Formulations of uricase nanoparticles are also provided. The nanoparticles can be formulated for administration to a subject, for example, as a pharmaceutical formulation. Therefore, pharmaceutical formulations including a plurality of functional recombinant uricase enzyme molecules, each independently having an amino acid sequence of any one or more of SEQ ID NOs:1-8 are described. Exemplary formulations include a solution, a dry powder, a tablet, micelles, colloids, nanodroplets, nano-structured hydrogel, nanocrystals, and a nanosuspension. Typically, the formulation includes a determined amount of uricase enzyme nanoparticles, in a form appropriate for a desired route of administration. Exemplary formulations of nanoparticles including uricase include liquids and dry powders. In some forms, the nanoparticles include active uricase enzyme in an amount from about 1% to about 100%, inclusive, from about 1% to about 80%, from about 1% to about 50%, preferably from about 1% to about 40% by weight, more preferably from about 1% to about 20% by weight, most preferably from about 1% to about 10% by weight. The ranges above are inclusive of all values from 1% to 100%. In some forms, the uricase agent to be delivered may be encapsulated within a nanoparticle and associated with the surface of the particle. For those forms where the uricase enzyme is associated with the surface of the particle, the percent loading may be higher since the amount of drug is not limited by the methods of encapsulation.

### 1. Liquid Formulations

In some forms, the nanoparticles are formulated as a liquid. Suitable liquid carriers include, but are not limited to, distilled water, de-ionized water, pure or ultrapure water, saline, and other physiologically acceptable aqueous solutions containing salts and/or buffers, such as phosphate buffered saline (PBS), Ringer's solution, and isotonic sodium chloride, or any other aqueous solution acceptable for administration to an animal or human.

Liquid formulations may include one or more suspending agents, such as cellulose derivatives, sodium alginate, polyvinylpyrrolidone, gum tragacanth, or lecithin. Liquid formulations may also include one or more preservatives, such as ethyl or n-propyl p-hydroxybenzoate.

Formulations may be prepared using one or more pharmaceutically acceptable excipients, including diluents, preservatives, binders, lubricants, disintegrators, swelling agents, fillers, stabilizers, and combinations thereof. Liquid formulations may also contain minor amounts of polymers, surfactants, or other excipients well known to those of the art. In this context, “minor amounts” means no excipients are present that might adversely affect the delivery of the nanoparticle compositions to organs or tissues, e.g., through circulation.

### 2. Dry Powder Formulations and Kit

In some forms, nanoparticles are formulated in dry powder forms as finely divided solid formulations. The dry powder components can be stored in separate containers or mixed at specific ratios and stored. In some forms, suitable aqueous and organic solvents are included in additional containers. In other forms, dry powder components, one or more solvents, and instructions on procedures to mix and prepare assembled nanostructures are included in a kit. Alternatively, stabilized, assembled particles, nanoparticles or bulk gel thereof are dried via vacuum-drying or freeze-drying, and suitable pharmaceutical liquid carrier can be added to rehydrate and suspend the assembled nanostructures or gel compositions upon use.

Dry powder formulations are typically prepared by blending one or more gelators, stabilizing agents, or active agents with one or more pharmaceutically acceptable carriers. Pharmaceutical carrier may include one or more dispersing agents. The pharmaceutical carrier may also include one or more pH adjusters or buffers. Suitable buffers include organic salts prepared from organic acids and bases, such as sodium citrate or sodium ascorbate. The pharmaceutical carrier may also include one or more salts, such as sodium chloride or potassium chloride. The dry powder formulations can be suspended in the liquid formulations to form nanoparticle solutions, and administered systemically or regionally using methods known in the art for the delivery of liquid formulations.

### 3. Injectable Formulations

In some forms, the assembled nanoparticles are formulated for parenteral delivery, such as injection or infusion, in the form of a solution or suspension. The formulation is preferably administered into the blood stream or, alternatively, directly to an organ or tissue in a subject. Formulations can be prepared as aqueous compositions using techniques is known in the art. Typically, such compositions can be prepared as injectable or infusible formulations, for example, solutions or suspensions; solid forms suitable for using to prepare solutions or suspensions upon the addition of a reconstitution medium prior to injection.

In some forms, the nanoparticles are formulated in a suitable carrier. A carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof.

In some forms, the nanoparticles are formulated to contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s). Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzyl peroxide, benzethonium chloride, benzyl alcohol, cetylpyridinium chloride, chlorobutanol, phenol, phenylethyl alcohol, and thimerosal.

In some forms, the nanoparticles are formulated to be buffered to a pH, for example, pH 2, 3, 4, 5, 6, 7, 8, 9 or pH 10. In an exemplary form, the formulation is typically buffered to a pH of 3-8 for parenteral administration. Suitable buffers include, but are not limited to, phosphate buffers, acetate buffers, and citrate buffers.

In some forms, the nanoparticles are formulated to include one or more water soluble polymers. Water soluble polymers are often used in formulations for parenteral administration. Suitable water-soluble polymers include, but are not limited to, polyvinylpyrrolidone, dextran, carboxymethylcellulose, and polyethylene glycol.

In some forms, the nanoparticles are formulated to be a sterile injectable solution. Sterile injectable solutions can be prepared by incorporating the nanoparticles in the required amount in the appropriate solvent or dispersion medium with one or more of the excipients listed above, as required, followed by filtered sterilization. Generally, dispersions are prepared by incorporating the various sterilized gelators, stabilizing agents, and/or active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Formulations may be prepared as described in standard references such as “Pharmaceutical dosage form tablets,” eds. Liberman et. al. (New York, Marcel Dekker, Inc., 1989), “Remington—The science and practice of pharmacy,” 20th ed., Lippincott Williams & Wilkins, Baltimore, MD, 2000, and “Pharmaceutical dosage forms and drug delivery systems,” 6th Edition, Ansel et al., (Media, PA: Williams and Wilkins, 1995). These references provide information on excipients, materials, equipment and process for preparing tablets and capsules and delayed release dosage forms of tablets, capsules, and granules.

### III. Method of Making Uricase Nanoparticles

Methods of making nanoparticles including recombinant soluble uricase enzymes and formulations thereof for administration to a subject are provided. Preferably, the recombinant soluble uricase enzymes are prepared including ancestral genomic uricase-like amino acid sequences. Exemplary recombinant soluble uricase enzymes include polypeptides having an amino acid sequence including SEQ ID NOs:1-8. Therefore, in some forms, the methods make nanoparticles including a plurality of functional recombinant uricase enzyme molecules, each independently having an amino acid sequence of any one or more of SEQ ID NOs:1-8.

### A. Uricase Nanoparticles

In some forms, the methods produce nanoparticles that are tetrameric structures of a multiplicity of recombinant soluble uricase enzymes, optionally cross-linked for enhanced stability. The tetrameric particles of uricase molecules are generally formed via the self-assembly or association (non-covalently or covalently) between two or more uricase molecules.

These particles may be nanosized or have a size in the micrometer range. They are generally in the form of vesicles, nanodroplets, nano-structured hydrogel, nanocrystals, or nanosuspension. In some forms, the methods assemble nanoparticles including nine or more functional recombinant uricase enzyme molecules, each independently having an amino acid sequence of any one or more of SEQ ID NOs:1-8.

In one embodiment, the uricase nanoparticles are prepared according to the method described in Example 2.

### B. Virus-Like-Particles (VLPs) of Uricase

In some forms, the methods produce nanoparticles including recombinant soluble uricase enzymes encapsulated within, or associated with virus-like particles. Exemplary virus like particles include Leviviridae-derived capsids. In some forms, the methods prepare and isolate nanoparticles sized for uptake within the gut following oral administration.

Methods for preparing VLPs encapsulating cargo molecules are known in the art, for example, as described in Pokorski, et al., J. Am. Chem. Soc. 133, 9242-9245 (2011), which is incorporated herein by reference in its entirety.

In various forms, the nanoparticle can be engineered to encapsulate various copy numbers of the cargo enzyme. For example, in various forms, each nanoparticle includes between 1 and 500 copies of a cargo uricase enzyme. In a preferred form, each nanoparticle includes between 2 and 18 copies of the cargo uricase enzyme.

In some embodiments, uricase enzymes are entrapped inside the Qβ protein nanoparticles by using enzyme packaging methodology previously described (Fiedler, J. D. et al., Biomacromolecules 2018, 19, 3945-3957). Briefly, the gene for an uricase variant is encoded into a bicistronic-plasmid expression system for Qβ VLP expression and packaging. The methodology involves the fusion of the uricase N-terminally to the positively charged Rev peptide sequence and inserted into a compatible pCDF plasmid, also express Qβ coat protein (CP). Compatible T7 expression vectors drive expression of CP, Rev-tagged cargo uricase, and bifunctional mRNA. The Rev-tag binds to the α-Rev aptamer and Qβ genome packaging hairpin binds to the interior of the CP monomers, tethering the uricase to the interior of the VLP. Therefore, transformation of this plasmid and expression in BL21 (DE3) E. coli yielded VLPs encapsidating the Rev-tagged uricase enzyme.

### 1. RNA-Directed Encapsulation Into Viral Capsids

In some forms, the methods utilize RNA-directed encapsulation of molecules into viral capsid proteins. Methods for RNA-directed encapsulation of cargo molecules into viral capsids are known in the art, for example, in US Publication No. 20130224828, the content of which is hereby incorporated by reference in its entirety. For example, in some forms, the methods of preparing the uricase nanoparticles include expression of expression vectors, the vectors together coding for all of the capsid protein, a tagged uricase cargo enzyme, and a bifunctional RNA including a tagged uricase cargo enzyme binding sequence and interior capsid wall binding sequence, in a Suitable expression system.

The viral capsid protein self-assembles into the protein nanoparticle upon expression. In an exemplary form, a tagged uricase enzyme can be a Rev-tagged uricase enzyme, produced in a similar manner from a suitably engineered plasmid in an organism such as E. coli, the plasmid containing a coding sequence along with the arginine-rich Rev tag sequence, using suitable promoters and the like, as is well known in the art. For example, the tagged uricase enzyme binding sequence of the bifunctional RNA can include a Rev-binding sequence, an interior capsid wall binding sequence including a viral capsid hairpin (hp) sequence, or both. The expression system can be any suitable living organism. In some forms, the methods provide a first plasmid coding for the capsid protein and the bifunctional RNA, and a second plasmid coding for the tagged uricase enzyme. In some forms the plasmid coding the uricase enzyme plus peptide tag sequence can be engineered to include coding sequences for any suitable enzyme of known sequence, combined with any suitable peptide tagging sequence. The cargo enzyme can be selected to catalyze a suitable reaction of interest, for example a hydrolytic reaction, such a cleavage of a peptide or a phosphate group. Accordingly, in some forms the plasmid is engineered to include a coding sequence for the peptide tag sequence in conjunction with a hydrolase or a phosphatase, such as a peptidase or a luciferase, respectively. In various forms, a first plasmid includes capsid protein RNA, a Rev aptamer disposed upstream of the ribosome binding site and the QB hairpin disposed immediately downstream of the stop codon. In a specific form, the first plasmid is a ColEl-group plasmid.

a. Encapsulation within Qβ Capsids

In some forms, the capsid protein is a Qβ capsid protein, which can be produced by expression of the Qβ coding sequence engineered in a plasmid in an organism, such as E. coli, using suitable promoters and the like.

In an exemplary form, example, the first plasmid can code for a Qβ capsid protein and an RNA containing a Qβ hairpin binding sequence; the second plasmid can code for a Rev-tagged uricase enzyme. The plasmids can contain suitable promoters, stop codons, and the like, as are well known in the art.

Therefore, in an exemplary form, VLPs are formed in a host bacterial system by mixing together the required nucleic acid plasmids or other vectors together and co-transfecting into the same cell-line. Assembled VLPs can be extracted and purified from the cells following expression. Since enzyme packaging relies only on co-expression of the components in E. coli cells, different variants of uricase are used with little or no optimization required. Isolated yields of VLP-packaged enzymes typically range from 10-1,000 mg of purified particle per liter of E. coli expression culture (usually >100 mg/L), which can be done in CLEARCOLI® cells to eliminate the possibility of endotoxin contamination. This is therefore a scalable, safe, and modular technology, well suited to optimization for the proposed application. Packaging uricases into Qβ vesicles does not affected the enzymatic properties of the uricase, and urate substrate can penetrate vesicles to react with the enzyme. These complexes are highly stable at 4° and the enzymatic properties are constant for at least 16 months when stored at this temperature. In some forms, the methods include one or more steps to characterize the resulting nanoparticles. For example, in some forms, the methods determine the immunogenicity of the nanoparticles in a recipient. Methods for monitoring and characterizing an immune response in a subject following exposure to a foreign body are known in the art.

b. Encapsulation within PP7 Capsids

In some forms, the capsid protein is a PP7 capsid protein, which can be produced by expression of the PP7 coding sequence engineered in a plasmid in an organism, such as E. coli, using suitable promoters and the like. Therefore, in some forms the methods encapsulate uricase enzymes within PP7 viral capsids, to form a PP7 VLP encapsulating uricases. Methods of engineering PP7 capsid proteins and engineering VLPs from PP7 capsids are known in the art, for example, as described in Zhao, et al., ACS Nano 2019, 13, 4, 4443-4454, which is incorporated herein by reference in its entirety.

c. Modification of Uricase VLPs

In some forms, the methods modify the viral capsid proteins and/or intact VLPs, to provide modified uricase VLPs. Methods for the modification of the VLPs by attachment of molecules to VLP capsids are also known, for example, as described in Crooke et al., ACS Appl. Bio Mater. 2, 93-103 (2019), which is incorporated herein by reference in its entirety.

### C. Methods of Formulating Uricase Nanoparticles

Preparation of nanosuspension is simple and applicable to water insoluble drugs. A nanosuspension provides improved solubility and bioavailability, as well as alters the pharmacokinetics of drug and thus improves drug safety and efficacy.

Other techniques for making particles include solvent evaporation, solvent removal, spray drying, phase inversion, low temperature casting, and nanoprecipitation. Suitable methods of particle formulation are briefly described below in the Examples. Pharmaceutically acceptable excipients, including pH modifying agents, disintegrants, preservatives, and antioxidants, can optionally be incorporated into the particles during particle formation.

### IV. Methods of Using Uricase Nanoparticles

Methods of using the uricase nanoparticles and compositions thereof are provided. Typically, the methods administer uricase nanoparticles to reduce or remove an amount of uric acid within a biological system, for example, to reduce the amount of uric acid within the blood of a human subject.

In some forms, the methods treat diseases and disorders associated with undesirable amounts of uric acid, or uric acid metabolites in a subject. The methods typically include administering to the subject an effective amount of a pharmaceutical formulation of uricase nanoparticles including a plurality of functional recombinant uricase enzyme molecules, each independently having an amino acid sequence of any one or more of SEQ ID NOs:1-8, to treat or prevent hyperuricemia in the subject. In preferred forms the methods treat or prevent one or more diseases or disorders associated with hyperuricemia. The methods administer pharmaceutical formulations of uricase nanoparticles to the subject via a route selected from oral administration, intramuscular injection, intravenous injection, sub-cutaneous injection, and intra-articular injection. In some forms the formulation is administered in an amount effective to reduce uric acid in the blood of the recipient to an amount less than or equal to 6 mg/dL uric acid in blood.

### A. Methods of Treating Diseases

Methods of treating hyperuricemia in a subject are provided. Uric acid is present in blood, and is often considered an undesirable metabolic “waste product” in humans. Excess uric acid is created when the body breaks down purines, and dissolves in the blood, passes through the kidneys and leaves the body in urine. Food and drinks high in purines also increase the level of uric acid. If too much uric acid stays in the body hyperuricemia will occur. Hyperuricemia can cause crystals of uric acid (or urate) to form, which can settle in the joints and cause gout. They can also settle in the kidneys and form kidney stones. If untreated, high uric acid levels may eventually lead to permanent bone, joint and tissue damage, kidney disease and heart disease. Research has also shown a link between high uric acid levels and type 2 diabetes, high blood pressure, and fatty liver disease.

Therefore, methods for reducing the amount of uric acid in a subject, including administering to the subject an effective amount of uricase nanoparticles to reduce the amount of uric acid in the subject are described. An exemplary method of treating, retarding development of, or preventing development of hyperuricemia in a subject includes administering to the subject in need thereof an effective amount of a pharmaceutical formulation including a plurality of functional recombinant uricase enzyme molecules, each independently having an amino acid sequence of any one or more of SEQ ID NOs:1-8 to retard development of, or prevent development of hyperuricemia in the subject.

In some forms, the methods treat diseases and disorders associated with Hyperuricemia, including Gout, hypertension, and renal disease. In some forms, the methods treat or prevent Tumor Lysis Syndrome (TLS), as well as Lesch-Nyhan disease are provided. Uric acid has also been recognized as a predictive marker for cardiovascular disease, hypertension, and renal disease.

All the methods described can also include the step of identifying and selecting a subject in need of treatment, or a subject who would benefit from administration with the uricase nanoparticles. In some forms, the subject has been medically diagnosed as having a hyperuricemia, Lesch-Nyhan disease, or a disease or disorder associated with hyperuricemia by exhibiting clinical (e.g., physical) symptoms of a disease. In other forms, the subject has been medically diagnosed as having a predisposition hyperuricemia, or to be at risk of a disease or disorder associated with hyperuricemia by exhibiting clinical (e.g., physical) symptoms, which are indicative of an increased risk or likelihood of developing hyperuricemia, or a disease or disorder associated with hyperuricemia. Therefore, in some forms, formulations of the disclosed uricase nanoparticles are administered to a subject prior to a clinical diagnosis of hyperuricemia, or a disease or disorder associated with hyperuricemia. In preferred forms, the methods administer uricase nanoparticles to a subject via oral administration, or via sub-cutaneous or intravenous injection.

In other forms, the methods induce or increase the expression or production of functional uricase enzyme in a subject in need thereof. For example, in some forms, methods of inducing or increasing uricase enzyme in a subject in need thereof include administering to the subject an effective amount of a gene editing system and a donor sequence encoding one or more uricase enzymes. Typically, the gene editing system is a CRISPR/Cas system. An exemplary CRISPR/Cas system includes (a) a single-guide RNA (sgRNA) that targets the human AAVS1 locus, and (b) a Cas nuclease or nickase. In an exemplary form, the one or more uricase enzymes are an ancestral form of a human uricase or a variant thereof.

### 1. Diseases to be Treated

In general, the compositions and methods of treatment thereof are useful in the context of treating and preventing Hyperuricemia in a subject. The compositions can also be used for treatment of other diseases and disorders including Gout, hypertension, and renal disease. In some forms, the methods treat or prevent Tumor Lysis Syndrome (TLS), as well as Lesch-Nyhan disease are provided

In some forms, the subject to be treated is a human. All the methods described can include the step of identifying and selecting a subject in need of treatment, or a subject who would benefit from administration with the described compositions.

a. Gout

In some forms, the methods administer uricase nanoparticles for the treatment or prevention of gout. Gout occurs where uric acid crystallizes in the tissues and is the most common inflammatory arthritis related to elevated uric acid levels. Recent reports estimate a gout incidence of 0.58-2.89 per 1,000 persons per year, is more prevalent in men than in women and increases in age. Gout is a form of arthritis that can be very painful, and includes gout flare (acute arthritis), chronic gouty arthritis, and tophaceous gout. Symptoms of gout include inflammation, pain and swelling. Acute gouty attack is usually monoarthritic that peaks within hours to severely inflamed joint with cardinal signs of inflammation including redness, hotness, tenderness, swelling and loss of function. In large joints such as knees and ankles, skin signs are infrequent, but swelling and pain can be intense.

Methods of treating gout include administering a subject in a need thereof an effective amount of nanoparticles including uricase to treat or prevent gout, including gout flare, chronic gouty arthritis and tophaceous gout. In some forms, the methods reduce or prevent one or more physiological markers of gout, including the amount of uric acid in the blood, and/or the presence of monosodium urate (MSU) crystals in joints, bones, or soft tissues. In other forms, the methods reduce or prevent one or more symptoms of gout in a tissue or organ of a subject, including inflammation (redness, hotness, tenderness, swelling), pain, and loss of function in the tissue or organ of the subject.

In other forms, the methods administer uricase nanoparticles for the treatment or prevention of one or more illnesses related to gout. Exemplary illnesses related to gout include erectile dysfunction, atrial fibrillation, obstructive sleep apnea, osteoporosis, and venous thromboembolism.

b. Hypertension

In some forms, the methods administer uricase nanoparticles for the treatment or prevention of Hypertension. Hyperuricemia occurs in 25-50% of hypertensive patients, and in up to 90% of those with new onset hypertension. Some studies suggest that high levels of uric acid confer a risk equivalent to the risk cholesterol poses for cardiovascular events. The methods typically include administering to a subject in a need thereof an effective amount of uricase nanoparticles to treat or prevent Hypertension. In some forms, the methods reduce or prevent one or more physiological markers of hypertension in a subject in need thereof. For example, in some forms, the methods reduce or inhibit one or more plasma markers including hs-CRP, Hcy, SUA, HbA1c and NT-proBNP in a subject diagnosed with, or at risk of hypertension associated with increased uric acid. In other forms, the methods reduce or prevent one or more symptoms of uric-acid induced hypertension in a subject in need thereof, including severe headache, nosebleed, Fatigue or confusion, vision problems, chest pain, difficulty breathing, irregular heartbeat and blood in the urine.

c. Renal Disease

In some forms, the methods administer uricase nanoparticles for the treatment or prevention of Renal disease. Hyperuricemia is a reported risk factor for the progression of renal disease; over 95% of patients with gout have interstitial renal disease at autopsy. Blood is filtered through the kidneys and uric acid can build up and form urate crystals (kidney stones). As the urate crystals pass through the kidneys, they can cause damage and scars. This kidney damage is thought to lead to kidney disease and failure over time. Most recently, uric acid has been shown to directly cause acute inflammation by stimulating the production of IL-1β in the inflammasome. Therefore, methods of treating renal diseases include administering to a subject in a need thereof an effective amount of uricase nanoparticles to reduce or prevent one or more symptoms of renal disease. Symptoms of renal diseases include a reduced amount of urine, swelling of the legs, ankles, and feet from retention of fluids caused by the failure of the kidneys to eliminate water waste, shortness of breath, excessive drowsiness or fatigue, blood in urine, foamy urine and itchy skin.

d. Tumor Lysis Syndrome (TLS)

In some forms, the methods administer uricase nanoparticles for the treatment or prevention of Tumor Lysis Syndrome (TLS). Tumor Lysis Syndrome (TLS) occurs when a large number of cancer cells die within a short period and release their contents into the blood. Generally, 1-5 days after chemotherapy starts, tumor lysis may occur, resulting in accumulation of uric acid crystals in the kidneys due to the release of massive amounts of purine DNA from lysed cells during cancer treatments. If cancer cells break down so quickly that the kidneys can't remove these substances from the blood, it can lead to tumour lysis syndrome (TLS). TLS is a group of problems with blood levels, including high levels of uric acid (hyperuricemia), potassium (hyperkalemia) and phosphate (hyperphosphatemia), and low levels of calcium (hypocalcemia). TLS is potentially life-threatening. If it is not controlled, abnormal blood levels can cause a variety of problems. Uric acid can be deposited in the joints, causing a painful gout-like condition. A buildup of uric acid in the kidneys can cause damage and stones to form. High phosphate levels can also damage the kidneys and lead to kidney failure. Abnormal levels of potassium and calcium can affect heart rhythm and lead to neurological changes such as weakness, irritability and confusion. TLS can lead to acute kidney injury, fatal arrhythmia, and death. The methods typically include administering a subject in a need thereof an effective amount of uricase nanoparticles to treat or prevent Tumor Lysis Syndrome in the subject. In some forms, the methods reduce or prevent one or more symptoms of TLS in a subject, including nausea, vomiting, diarrhea, muscle cramps or twitches, weakness, numbness or tingling sensations, fatigue, decreased urination, irregular heart rate, confusion, restlessness, irritability, delirium or hallucinations and seizures.

e. Lesch-Nyhan disease

In some forms, the methods administer uricase nanoparticles for the treatment or prevention of Lesch-Nyhan disease are provided. Lesch-Nyhan syndrome (LNS) is a rare, inherited disorder caused by a deficiency of the enzyme hypoxanthine-guanine phosphoribosyltransferase (HPRT). LNS is an X-linked recessive disease.

The methods typically include administering a subject in a need thereof an effective amount of nanoparticles including uricase to treat or prevent one or more symptoms of Lesch-Nyhan disease, including impaired kidney function, acute gouty arthritis, and neurological indications, including self-mutilating behaviors such as lip and finger biting and/or head banging, involuntary muscle movements, and cognitive impairment.

### 2. Treatment Regimens

A treatment regimen can include one or multiple administrations of the uricase nanoparticles and formulations thereof for achieving a desired physiological change, including administering to an animal, such as a mammal, especially a human being, an effective amount of the compositions to treat the disease or symptom thereof, or to produce the physiological change. In preferred forms, the desired physiological change is the reduction in the amount of uric acid in the blood and/or tissues and organs of the subject.

a. Dosage and Effective Amounts

A therapeutically effective amount of uricase nanoparticles used in the treatment of diseases and disorders associated with hyperuricemia are typically sufficient to reduce or alleviate one or more symptoms of the diseases and disorders associated with Hyperuricemia. Symptoms of diseases and disorders associated with Hyperuricemia may be physical, such as gout, or biological such as increased uric acid in the blood. Accordingly, the amount of uricase nanoparticles can be effective to, for example, treat or prevent one or more symptoms of gout, or reduce uric acid in the blood, tissue or organs. Preferably the uricase nanoparticles are delivered systemically, for example, orally or via sub-cutaneous or intravenous injection. Preferably the uricase nanoparticles do not target or otherwise modulate other metabolic processes or metabolic products, or do so at a reduced level compared to that relating to uric acid. In some forms, the uricase nanoparticles reduce Hyperuricemia, or uric acid crystals within the joints and other tissues, or a combination thereof. In some forms, the uricase nanoparticles are administered in an effective amount to prevent hyperuricemia, or one or more diseases or disorders associated with hyperuricemia in a subject at risk of hyperuricemia.

The actual effective amounts of uricase nanoparticles can vary according to factors including the specific uricase nanoparticles administered, the particular composition formulated, the mode of administration, and the age, weight, condition of the subject being treated, as well as the route of administration and the disease or disorder.

The therapeutic result of the uricase nanoparticles can be compared to a control. Suitable controls are known in the art. A typical control is a comparison of a condition or symptom of a subject prior to and after administration of the uricase nanoparticles. The condition or symptom can be a biochemical, molecular, physiological, or pathological readout. For example, the effect of the uricase nanoparticles on a particular symptom, pharmacologic, or physiologic indicator can be compared to an untreated subject, or the condition of the subject prior to treatment. In some forms, the symptom, pharmacologic, or physiologic indicator is measured in a subject prior to treatment, and again one or more times after treatment is initiated. In some forms, the control is a reference level, or average determined based on measuring the symptom, pharmacologic, or physiologic indicator in one or more subjects that do not have the disease or condition to be treated (e.g., healthy subjects). In some forms, the effect of the treatment is compared to a conventional treatment that is known the art.

In some forms, the effective amount of uricase nanoparticles causes little or no killing of cells within the subject, and preferably little or no inhibition of metabolism in cells. It is particularly preferred that the composition have little or no effect on immune cells.

In some forms, dosages of uricase nanoparticles are administered once, twice, or three times daily, or every other day, two days, three days, four days, five days, or six days to a human. In some forms, dosages of uricase nanoparticles are administered about once or twice every week, every two weeks, every three weeks, or every four weeks. In some forms, dosages are administered about once or twice every month, every two months, every three months, every four months, every five months, or every six months.

In some forms, the regimen includes one or more cycles of a round of therapy with uricase nanoparticles followed by a drug holiday (e.g., no uricase nanoparticles). The round of the therapy can be, for example, any of the administrations discussed above. Likewise, the drug holiday can be 1, 2, 3, 4, 5, 6, or 7 days; or 1, 2, 3, 4 weeks, or 1, 2, 3, 4, 5, or 6 months.

In particular forms, the subject is administered a dosage of between about 0.1 mg/kg body weight and 100 mg/kg body weight, inclusive, of uricase nanoparticles. In some forms, the subject is administered a dosage of between about 0.1 mg/kg body weight and 10 mg/kg body weight, inclusive, of uricase nanoparticles. In some forms, the subject is administered a dosage of between about 0.2 mg/kg body weight and 10 mg/kg body weight, inclusive, of uricase nanoparticles. In some forms, the subject is administered a dosage of between about 0.3 mg/kg body weight and 10 mg/kg body weight, inclusive, of uricase nanoparticles. In some forms, the subject is administered a dosage of between about 0.4 mg/kg body weight and 10 mg/kg body weight, inclusive, of uricase nanoparticles. In some forms, the subject is administered a dosage of between about 0.5 mg/kg body weight and 10 mg/kg body weight, inclusive, of uricase nanoparticles. In some forms, the subject is administered a dosage of between about 1.0 mg/kg body weight and 10 mg/kg body weight, inclusive, of uricase nanoparticles. In some forms, the subject is administered a dosage of between about 1.0 mg/kg body weight and 5 mg/kg body weight, inclusive, of uricase nanoparticles. Particular dosage regimens include, for example, one or more cycles in which the subject is administered the uricase nanoparticles on each of two, three, four, five, six or seven days, weeks or months in a row, followed by a one, two, three, four, five, six or seven-day, week, or month drug holiday.

In the most preferred forms, methods of using the uricase nanoparticles lead to direct or indirect reduction in the level of uric acid or uric acid crystals in the blood or tissue or organs and/or direct or indirect inhibition of the production of uric acid or equivalents, or the direct or indirect reduction of uric acid, or uric acid crystals in the blood or tissue or organs. In some forms, the effective amount of uricase nanoparticles is suitable for achieving a specific target amount of uric acid in the blood. In an exemplary form, the effective amount of uricase nanoparticles is suitable for achieving a value of <6 mg/dL uric acid in the blood.

### B. Combination Therapies and Procedures

The uricase nanoparticles can be administered alone or in combination with one or more conventional therapies, for example, a conventional hyperuricemia, or gout therapy. In some forms, the conventional therapy includes administration of uricase nanoparticles in combination with one or more additional active agents. The combination therapies can include administration of the uricase nanoparticles and other active agents together in the same admixture, or in separate admixtures. Therefore, in some forms, the pharmaceutical composition includes uricase nanoparticles and one, two, three, or more additional active agents. The additional active agent(s) can have the same, or different mechanisms of action. In some forms, the combination results in an additive effect on the treatment of hyperuricemia, or gout. In some forms, the combinations result in a more than additive effect on the treatment of the disease or disorder.

The additional therapy or procedure can be simultaneous or sequential with the combination therapy. In some forms the additional therapy is performed between drug cycles or during a drug holiday that is part of the compositions dosage regime. For example, in some forms, the additional therapy or procedure is surgery, a radiation therapy, or chemotherapy.

Additional therapeutic agents include conventional therapeutics such as nonsteroidal anti-inflammatory drugs (NSAID), corticosteroids, and colchicine. Treatment with xanthine oxidase inhibitors (XOI) or uricosuric drugs is indicated for patients with a recurrent or severe course of gout. Therefore, in some forms, the additional therapeutic agents include xanthine oxidase inhibitors (XOI) and/or uricosuric drugs. In other forms, the additional active agent is another drug directly or indirectly targeting uric acid, such as an additional uricase enzyme, or associated enzyme-based therapeutic drug. Exemplary drugs targeting uric acid and uric acid metabolism include Uricozyme and Rasburicase/Elitek, and Pegloticase KRYSTEXXA®.

The present invention will be further understood by reference to the following non-limiting examples.

### EXAMPLES

### Example 1: Using CRISPR-Cas9 Genomic Engineering Tools to Identify Functional Ancestral Uricase

### Materials and Methods

### Guide RNA Sequences

(SEQ ID NO: 9)
gRNA1: 5′ACCCCACAGTGGGGCCACT3′
(SEQ ID NO: 10)
gRNA2: 5′CTAGGGACAGGATTGGTGAC3′

### Cell Culture

Human embryonic kidney cells (HEK293) were purchased from ATCC. HEK293 cells were cultured in Dulbecco's Modified Eagle's Medium (DMEM, Corning) supplemented with 10% fetal bovine serum (FBS, Corning) and 1% antibiotic-antimycotic solution (Gibco). Cells were passaged ˜three times per week and maintained in a humidified incubator at 37° C. with 5% CO₂.

### Construction of CRISPR and Donor Plasmids

To construct the pU6-(Bbs1) CBh-Cas9-T2A-GFP-p2A-Ad4E4orf6 plasmid (CRISPR plasmid), we digested px458 (Addgene, #48138) and pU6-(BbsI) CBh-Cas9-T2A-mCherry-P2A-Ad4E4orf6 (Addgene, #64222) with FseI/BsrGI (New England Biolabs). The GFP fragment was ligated into pU6-(BbsI) CBh-Cas9-T2A-_-P2A-Ad4E4orf6 vector from which the RFP/mCherry gene was removed. AAVS1 target sequences of gRNA used in a study were previously published (Balico et al., Mol Ther Nucleic Acids. 2021 Aug. 19; 25:578-584. doi:10.1016/j.omtn.2021.08.002). gRNA oligonucleotides were annealed and cloned into BbsI sites to generate both the gRNA1-CRISPR and gRNA2-CRISPR plasmid.

To generate the AAVS1-SA-T2A-RFP-T2A-AncUOX-SV40polyAsignal donor plasmid, the coding regions for RFP-T2A-AncUOX-SV40polyAsignal were synthesized as mammalian codon-optimized sequences by GeneScript (Thermo Scientific), amplified and cloned into AAVS1 SA-T2A-puro pA donor (Addgene #22075) by Gibson Assembly (New England Biolabs). Primers used are listed in Table 1.

TABLE 1
Primers used in this study.

| Primer Name | Sequence |
|---|---|
| gRNA1-F | 5′CACCGACCCCACAGTGGGGCCACTA3′ |
| | (SEQ ID NO: 11) |
| gRNA1-R | 5′AAACTAGTGGCCCCACTGTGGGGTC3′ |
| | (SEQ ID NO: 12) |
| gRNA2-F | 5′CACCGTCACCAATCCTGTCCCTAG3′ |
| | (SEQ ID NO: 13) |
| gRNA2-R | 5′AAACCTAGGGACAGGATTGGTGAC3′ |
| | (SEQ ID NO: 14) |
| Fragment | 5′ATCCCGGCCCTAGGCTCGAGATGGCCAGCAGCGA |
| Gibson | GGACGT3′ (SEQ ID NO: 15) |
| Assembly-F | |
| Fragment | 5′TCACCAATCCTGTCCCTAGTTAAGATACATTGATG |
| Gibson | AGTTT3′ (SEQ ID NO: 16) |
| Assembly-R | |
| Vector | 5′ AAACTCATCAATGTATCTTAACTAGGGACAGGAT |
| Gibson | TGGTGA3′ (SEQ ID NO: 17) |
| Assembly-F | |
| Vector | 5′ACGTCCTCGCTGCTGGCCATCTCGAGCCTAGGGCC |
| Gibson | GGGAT3′ (SEQ ID NO: 18) |
| Assembly-R | |
| F1 | 5′GGCAGCCTGTGCTGACCCATGCAGTC3′ |
| | (SEQ ID NO: 19) |
| R1 | 5′TAAGATACATTGATGAGTTTGGACAAACCA3′ |
| | (SEQ ID NO: 20) |
| F2 | 5′AACCCTTACGGCAAGATCACCG3′ |
| | (SEQ ID NO: 21) |
| R2 | 5′CCACAGTTGGAGGAGAATCC3′ |
| | (SEQ ID NO: 22) |

### Transfection and Fluorescence-Activated Cell Sorting (FACS)

HEK293 were transfected with TransfeX Reagent (ATCC) following the manufacturer's protocol. Briefly, 24 h prior to transfection, 1×10⁶ cells were plated into a single well of a 6-well plate. For each transfection, 2 μg of gRNA1- or gRNA2-CRISPR plasmid was mixed with 4 μg donor plasmid in Opti-MEM (Invitrogen) and 12 μL of TransfeX. After 15 minutes of incubation at room temperature, DNA complexes were mixed carefully into the wells containing the cells. Transfected cells were passaged at least twice before FACS. Bulk cell sorting was performed on a BD FACS Aria II (BD Biosciences), the RFP-positive cells were sorted into FACS tubes with DMEM supplemented with 20% FBS and 1% antibiotic-antimycotic solution. After sorting, cells were cultured until confluence.

### Genomic DNA Extraction, Genotyping PCR and Sequencing

Genomic DNA from sorted and cultured wild-type and RFP-positive cells was extracted using DNeasy Blood & Tissue kit (QIAGEN) according to the manufacturer's protocol. To detect donor integration into AAVSI locus, genomic DNA was amplified using Q5 Hot Start High-Fidelity Master Mix (New England Biolabs) with primers described. PCR products were purified using QIAquick PCR Purification kit (QIAGEN) and sequenced.

### Western Blot Analysis

The wild-type and RFP-positive cells were lysed in denaturing lysis buffer (50 mM This-HCl pH 7.5, 1% SDS, 5 mM EDTA, 10 mM BME, 1 mM PMSF and 15U/mL DNase) in the presence of protease/phosphatase inhibitor cocktails (Thermo Scientific), mix by vortexing for 3 seconds at maximum speed and samples were heated at 95° C. for 5 minutes. Suspension was then diluted in NP-40 lysis buffer (50 mM Tris-HCl pH 7.4, 150 mM NaCl, 1% NP-40 and 5 mM EDTA) and mixed gently. The lysed suspension was passed through a 26-gauage needle attached to a 1 mL syringe and incubated on ice for 5 minutes. Samples were centrifuged for 15 minutes at 16,000×g at 4° C. The supernatants were transferred to a fresh microcentrifuge tube and protein concentration were determined using BCA protein assay kit (Pierce, Thermo Scientific). 40 μg of total protein was boiled at 95° C. for 5 minutes and loaded into SDS-PAGE gels (Bio-Rad) and then transferred to PVDF membranes (Bio-Rad). Membrane was incubated with anti-uricase (sc-166070, Santa Cruz Biotechnology) or anti-catalase (#21260-1-AP, Proteintech-Thermo Scientific) or anti-b-actin (sc-47778, Santa Cruz Biotechnology). Membranes were incubated with HRP-conjugated secondary antibodies. Signal was detected after incubation with Clarity Western ECL Substrate kit (Bio-Rad) and recorded with a ChemiDoc Imaging System (Bio-Rad).

### Immunofluorescence and Confocal Microscopy

HEK293 RFP-positive and control wild-type cells were cultured under glass cover slips in 24-well plates (5×10⁴). To determine the co-localization of peroxisomes and AncUOX, cells were fixed, permeabilized, blocked and stained with anti-peroxisomal membrane protein 70 (PMP70) using SelectFX Alexa Fluor 488 Peroxisome Labeling Kit (Invitrogen, Molecular Probes) following the manufacturer's instructions. Cells were rinsed and then stained with anti-uricase (sc-166070, Santa Cruz Biotechnology) and secondary anti-mouse Alexa Fluor 647 (Thermo Fisher). Nuclei were stained with DAPI. Images were captured with 63X oil immersion objective by using a laser scanning confocal microscope LSM 700 (Zeiss).

### Uricase Activity

Uricase activity was measured by monitoring the decrease of absorbance at 293 nm due to the enzymatic oxidation of uric acid. 1 mM uric acid stock solution was freshly prepared in 0.1M sodium phosphate, buffer pH 7.4, and the solution was incubated at 37° C. for 5 minutes with shaking. The assays were performed in triplicate and the following uric acid concentrations: 100 μM, 200 μM, 400 μM and 600 μM diluted in 0.1 M sodium phosphate buffer, pH 7.4. Uric acid solutions were microscopically analyzed to confirm the absence of substantial uric acid crystallization. Cells were lysed with 1×Assay Buffer Uric Acid/Uricase Assay Kit (Cell Biolabs). Data were normalized by total protein concentration.

### Results

The progressive loss of uricase activity in the ape lineage may have provided a selective advantage to our ancestors (Johnson R J et al., Semin Nephrol 31, 394-399 (2011)). While several theories have been proposed, a recent hypothesis is that the loss of uricase may have aided the accumulation of fat stores in response to fructose, a major nutrient in fruits that were a primary staple of ancestral simians (Johnson R J et al., Evolutionary Anthropology 19, 250-257 (2010); Choi Y J et al., Lab Invest 94, 1114-1125 (2014)). Fructose increases hepatic fat stores due, in part, to its ability to generate uric acid during metabolism (Lanaspa M A et al., J Biol Chem 287, 40732-40744 (2012)) and the inhibition of uricase enhances fat accumulation in the liver in response to fructose (Tapia E et al., Am J Physiol Renal Physiol, (2012)).

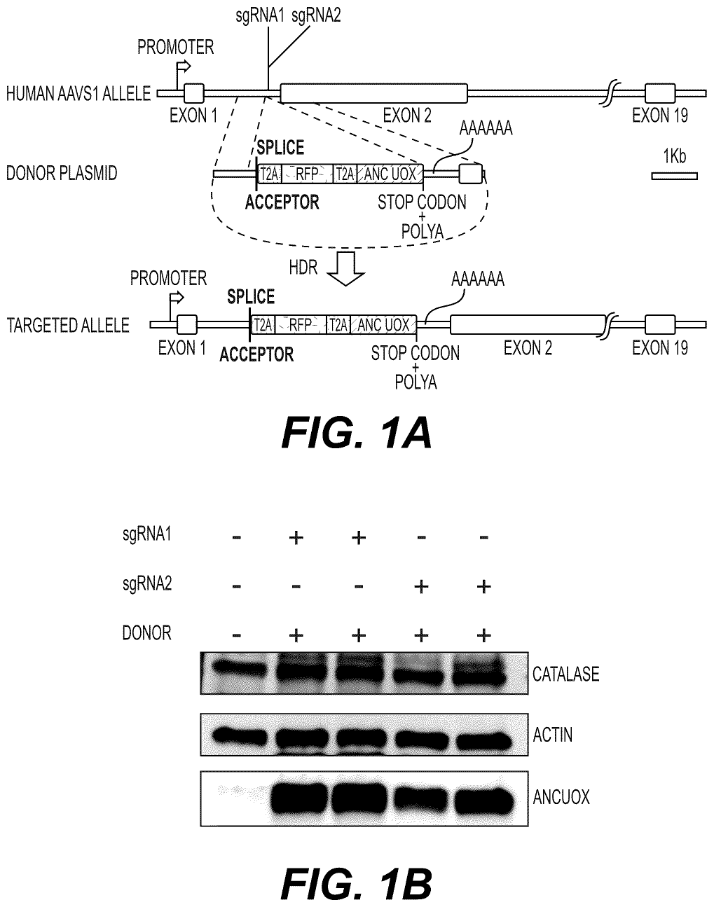

FIG. 1A shows a successful iteration of our CRISPR-uricase system that used two separate guide-RNAs. A donor fragment containing RFP and ancestral uricase An67 was genomically integrated into the AAVS1 location of HEK293 cells. PCR screening suggests that the donor integrated at only the AAVS1 location, no off-target insertion. Sequencing confirmed both donor integration and a CRISPR scar in the genomic DNA. As shown in the figure, donor DNA is transcribed and translated off the endogenous myosin gene at this locus. T2A self-cleaving protein domains are integrated to ensure both RFP and uricase proteins are liberated from the exon 1 open-reading-frame of the myosin phosphatase peptide after translation. AAVS1 locus is a focus of the biomedical community because integration is very specific (limited off-target integration), and disruption of the myosin phosphatase gene has minimal effect on human cells. A stop codon is engineered immediately after the last sense codon of uricase in order to ensure that the 3-amino acid peroxisomal tag (S-K-L) is completely accessible on the carboxyl terminus and thus uptake of the uricase by peroxisomes. Western blotting confirms that uricase is expressed in the engineered cells, but not in non-transfected cells (FIG. 1B). Immunofluorescence against DAPI (nucleus-specific), catalase (a peroxisome-specific protein) and uricase demonstrates that uricase has indeed localized to the peroxisome.

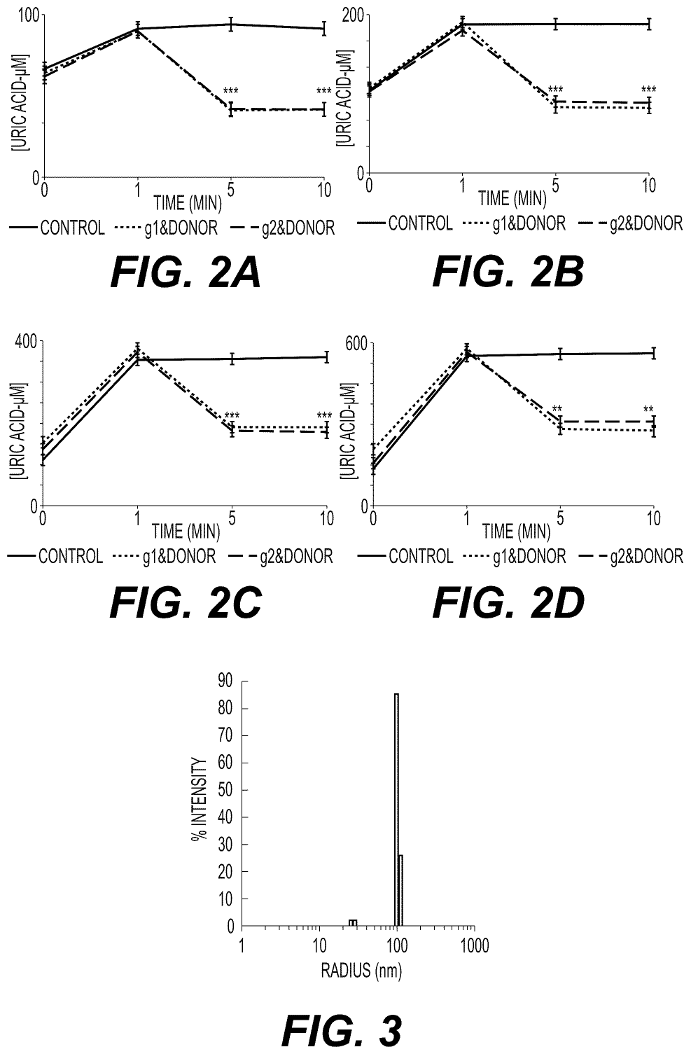

Control and uricase-positive cells were then subjected to uric acid exposure at 100, 200, 400, and 600 μM (FIGS. 2A-2D). Only uricase-positive cells lowered uric acid levels, and they achieved this quickly. It is noteworthy that even uricase-positive cells exposed to 600 μM can rapidly lower uric acid levels to almost pre-exposure levels because this concentration is the equivalent of 10 mg/dL in humans (clinical indicator of gout). The uricase enzyme suffers from product inhibition which may explain why cells exposed to high concentrations (400 and 600 μM) could not achieve pre-exposure levels even after two hours.

Data have shown certain tissue types transcribe uricase genes. In particular, four tissue types were grown and analyzed for uricase transcript level-three brain tissues (A172, H4, LN229) and one kidney tissue (HEK293). All four express a uricase-like protein (˜30 kDa) when exon 3 was mixed from the full-length uricase protein and if both premature stop codons were read through.

CRISPR-based uricase systems and/or engineered uricase proteins can provide both research and therapeutic applications. It has been demonstrated that CRISPR can be used to genomically integrate a functional uricase into human kidney cells. This is mostly proof-of-principle because uricase has its largest functional effect in the liver of most mammals-it is also expressed to a lower extent in kidneys as shown in some animals though (B. Farina, et al., Ital J Biochem 28, 270-279 (1979; R. Truszkowski, et al., Biochem J 29, 2787-2797 (1935)). Understanding the role of uricase in limiting fructose's ability to stimulate fat synthesis in the liver (most fat in humans is synthesized in the liver and adipose tissue) is of particularly interest, the uricase genome engineering system will be used in human liver cells and the broad-scale effects of uricase expression on fat synthesis and fat metabolism in hepatocytes will be investigated.

### Example 2: Production of Nanoparticles Formed from Ancestral

### Uricase Enzyme

To develop an enhanced delivery platform, recombinant ancient uricases were self-assembled into a variety of active homogeneous nanoparticle structures.

### Materials and Methods

The size of the nanoparticles can be adjusted by exposure to different pH, and determined by Dynamic Light Scattering (DLS) to elucidate a range of sizes from 1-1000 nm for the uricases. Since the particles are sensitive to fluctuations in pH, the ability to crosslink the structures with agents such as GTA, DTSSP and BS3 is contemplated. The cross-linked uricase nanoparticles will then be injected or gavaged into uricase knockout mice to determine any potential therapeutic utility.

### Results

In one formula, uricase was assembled into nanoparticles of approximately 100 nm in radius, to ˜94% homogeneity (FIG. 3, Table 2). This size of nanoparticle is especially ideal for delivery of therapeutic drugs to the digestive tract.

TABLE 2
Sizes of nanoparticles measured by DLS

| Peak | Radius (nm) | Mw-R (kDa) | % Mass |
|---|---|---|---|
| 1 | 28.00 | 8187 | 6.5 |
| 2 | 98.18 | 154,244 | 93.5 |

### Example 3: Production of Virus-Like Particles (VLPs) Encapsulating Ancestral Uricase Enzyme

In addition to developing nanoparticles composed solely of uricases packed into higher-order structures, uricases were package into viral-like particles (or vesicles) composed of the Qβ capsid protein.

A reliable method for encapsulating functional proteins and enzymes in stable and protective nanoparticles (virus-like particle, VLP), and for producing those particles in large quantities has been developed. Co-expression of a capsid protein, a cargo protein, and non-translated mRNA bearing aptamer sequences that bind to both protein types leads to efficient assembly of these three-component particles (see Fiedler, et al., Angew. Chem. Int. Ed. 49, 9648-9651 (2010)).

Since enzyme packaging relies only on co-expression of the components in E. coli cells, different variants of uricase can be used with little or no optimization required. Isolated yields of VLP-packaged enzymes typically range from 10-1000 mg of purified particle per liter of E. coli expression culture (usually >100 mg/L), which can be done in CLEARCOLI® cells to eliminate the possibility of endotoxin contamination. This is therefore a scalable, safe, and modular technology, well suited to optimization for the proposed application.

The Leviviridae-derived capsids (such as Qβ, PP7, and MS2 viruses) are stable toward thermal denaturation at temperatures up to 80-100° C., chaotropic agents, and to extremes of pH. These VLPs are also highly stable toward chemical manipulation of the capsid protein, giving rise to a variety of modifications that can be used to further stabilize the particles. For example, the multivalent attachment of polymers can render the particles nearly indestructible, as well as shield them from binding to undesired surfaces or proteins. Chemical reactions using small-molecule reagents also result in the modification of packaged enzymes, sometimes abrogating catalytic activity, but the attachment of large, branched molecules takes place exclusively on the VLP surface, keeping the entrained enzymes active.

It is therefore contemplated to impart desired properties to the VLP package (such as enhanced stability toward stomach fluid, anchoring on the intestinal mucosa, and immunological shielding) while retaining uricase activity. While the preliminary results have been generated using the Qβ particle, the even more robust PP7 platform will likely be used for animal experiments. The potential immunogenicity of these nanoparticles will be monitored in the knockout mice.

### Methods

### Cloning, Production, and Purification of Enzyme-Packaged VLPs

Bicistronic plasmids coding for both Qβ coat protein (CP) and Rev peptide-tagged enzymes in the pCDF-1b parent vector were used as previously described (Fiedler, J. D.; Fishman, M. R.; Brown, S. D.; Lau, J.; Finn, M. G., Multifunctional Enzyme Packaging and Catalysis in the Q beta Protein Nanoparticle. Biomacromolecules 2018, 19, 3945-3957). All sequences were verified by sequencing before expression. E. coli BL21 (DE3) (Biogen) cells harboring the appropriate plasmids were grown in either SOB (Amresco) supplemented with 20 mM magnesium sulfate and 50 μg/mL streptomycin. Starter cultures were grown overnight at 37° C. and used to inoculate larger expression cultures. Expression was induced with 1 mM IPTG when OD₆₀₀ reached approximately 1.0, and the induced culture was kept at room temperature for overnight expression. Cells were harvested by centrifugation in a JA-10 rotor at 6000 rpm, and the pellets were either processed immediately or stored at −80° C. The cell lysate was prepared by re-suspending the cell pellet with 100 mL of 100 mM phosphate buffer (pH 7.0) and sonicating at 30 W for 10 min with 5 s bursts and 5 s intervals. Cell debris was pelleted in a JA-17 rotor at 14000 rpm, and 0.265 gm/mL ammonium sulfate was added to the supernatant to precipitate the VLPs. The crude VLP pellet from precipitation was re-suspended in 3 mL of 100 mM phosphate buffer (pH 7.0). Organic extraction with 1:1 n-butanol:chloroform was performed to remove lipids and other cellular debris from VLPs. The aqueous layer containing VLPs were further purified by sucrose density ultracentrifugation (10-40% w/v). Particles were pelleted out by ultracentrifugation in a 70Ti rotor (Beckman) at 68000 rpm for 2 hours.

### Results

Up to 25 copies of 30-kD fluorescent proteins have been packaged into VLPs this way, as have functional RNA aptamers. Enzymes so entrained are significantly stabilized toward thermal and chaotropic denaturation, leading to enhanced, long-lived catalytic activity when the enzyme substrates and products can diffuse into and out of the capsid shell, such as uric acid.

Since enzyme packaging relies only on co-expression of the components in E. coli cells, different variants of uricase can be used with little or no optimization required. Isolated yields of VLP-packaged enzymes typically range from 10-1000 mg of purified particle per liter of E. coli expression culture (usually >100 mg/L), which can be done in CLEARCOLI® cells to eliminate the possibility of endotoxin contamination. This is therefore a scalable, safe, and modular technology, well suited to optimization for the proposed application. Packaging uricases into Qβ vesicles has not affected the enzymatic properties of the uricase, urate substrate can penetrate vesicles to react with the enzyme. These complexes are highly stable at 4° and the enzymatic properties are constant for at least 16 months when stored at this temperature.

### Example 4: Virus-Like Particles (VLPs) Encapsulating Ancestral Uricase Enzyme are Effectively Administered Via Oral Delivery

### Methods

Uricase knockout mice were administered An67 (AncUOX) encapsulated in a vesicle of Qβ capsid protein, termed Qβ-AncUOX, or just the vesicle itself, termed Qβ-empty, via oral gavage twice daily for 29 days starting at 3 weeks of age, weaned off allopurinol at 2 weeks of age to ensure mice were in a diseased state without chronic kidney failure.

The ratio of urine uric acid to creatinine was monitored once a week for six weeks.

### Results

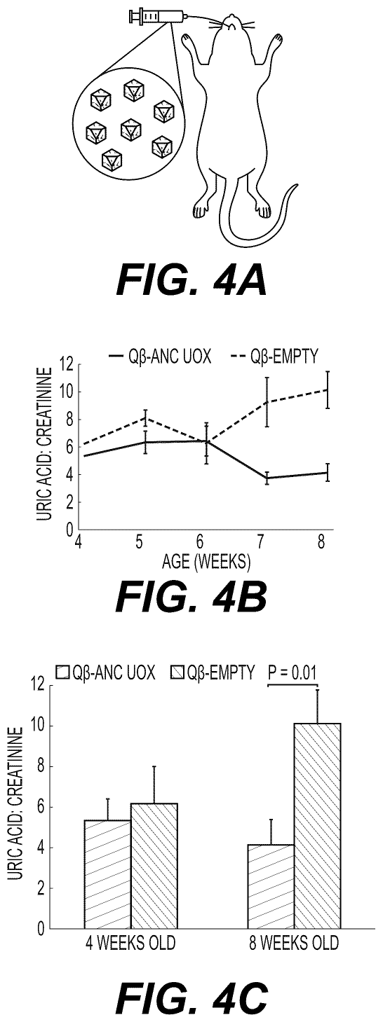

As set forth in FIGS. 4A-4C, WT mice had a ratio ≤1.0. Data are presented as mean±SEM for Qβ-AncUOX (solid line) and Qβ-empty (dashed line), n=4 (two females and two males).

FIG. 4B demonstrates the difference in the ratio of urine uric acid to creatinine between Qβ-AncUOX (black) and Qβ-empty (gray) is shown at the beginning of the experiment (week 2) and at the end of the experiment (week 6).

Data are presented as mean±SEM, n=4 in each group (two females and two males). Significance was evaluated using a student t-test in SPSS Statistics. Kidneys were harvested and stained for by IDEXX Laboratories.

### Example 5: Improved Thermostability of Ancestral Uricase Mutant Methods

To measure the thermostability of uricases, the enzyme samples were incubated in a water bath at 30° C., 37° C. and 42° C. At indicated time points, 1 μL of 1 mg/mL purified uricase sample was added to 100 μM uric acid dissolved in 1X PBS. The decrease in uric acid concentration was monitored at 293 nm and 37° C. The exponential regression (one phase decay) was performed with Graphpad Prism 9.

### Results

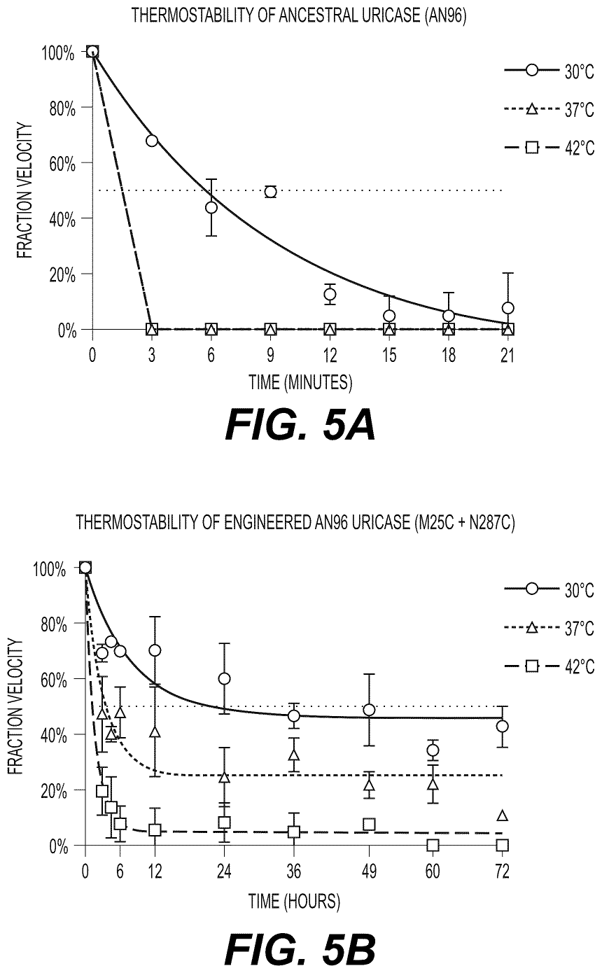

As set forth in FIGS. 5A-5B, the thermostability of an ancestral uricase (An96 having SEQ ID NO:8) was significantly improved by mutating two amino acid positions M25C and N287C.

The invention will be further understood by virtue of the following numbered paragraphs.

1. A nanoparticle comprising
a plurality of functional recombinant uricase enzyme molecules each independently having an amino acid sequence of any one or more of SEQ ID NOs:1-8,
wherein the nanoparticle has a diameter of between about 50 nm and 1,000 nm, inclusive, as determined by Dynamic Light Scattering (DLS) analysis.

2. The nanoparticle of paragraph 1, wherein the nanoparticle comprises a nanostructure consisting of some or all of the functional recombinant uricase enzyme molecules,
wherein the nanostructure is formed by non-covalent interactions between nine or more of the functional recombinant uricase enzyme molecules.

3. The nanoparticle of paragraph 1, wherein the nanoparticle comprises a nanostructure, wherein the nanostructure consists of
(a) some or all of the functional recombinant uricase enzyme molecules; and
(b) one or more cross-linker molecules,
wherein the nanostructure is formed by assembly of nine or more of the functional recombinant uricase enzyme molecules, wherein the one or more cross-linker molecules cross-link two or more of the functional recombinant uricase enzyme molecules within the nanostructure.

4. The nanoparticle of paragraph 2 or 3, wherein the nanostructure is formed by self-assembly of the functional recombinant uricase enzyme molecules.

5. The nanoparticle of paragraph1, wherein the nanoparticle is a virus-like-particle (VLP), comprising
(a) the functional recombinant uricase enzyme molecules; and
(b) viral capsid proteins,
wherein the functional recombinant uricase enzyme molecules are encapsulated within the viral capsid proteins.

6. The nanoparticle of paragraph 5, wherein the viral capsid proteins are from a virus selected from the group consisting of bacteriophage Q-beta, bacteriophage PP7, and bacteriophage MS.

7. The nanoparticle of paragraph 5 or 6, wherein the VLP encapsulates between one and one hundred functional recombinant uricase enzyme molecules.

8. The nanoparticle of paragraph 7, wherein the VLP encapsulates between ten and thirty functional recombinant uricase enzyme molecules.

9. The nanoparticle of paragraph 8, wherein the VLP encapsulates twenty-five functional recombinant uricase enzyme molecules.

10. The nanoparticle of any one of paragraphs 5 to 9, wherein the VLP is modified by attachment of one or more moieties to an external face of one or more of the viral capsid proteins.

11. The nanoparticle of paragraph 10, wherein the one or more moieties are independently selected from the group consisting of a protein, a nucleic acid, a carbohydrate, a polymer, a lipid, a small molecule, a secondary nanoparticle, a microparticle, a cell, and a virus.

12. The nanoparticle of paragraph 10 or 11, wherein one or more of the moieties are functional moieties, wherein the functional moieties are independently selected from the group consisting of a targeting moiety, a fluorescent label, a therapeutic agent and a cross-linking agent.

13. The nanoparticle of any one of paragraphs 1-12, wherein the nanoparticle encapsulates one or more additional active agents selected from the group consisting of therapeutic agents, diagnostic agents, nutraceutical agents, and enzyme catalysts.

14. The nanoparticle of any one of paragraphs 1-13, wherein the diameter of the nanoparticle is between about 75 nm and about 150 nm, as measured by DLS analysis.

15. The nanoparticle of any one of paragraphs 1-14, wherein the diameter of the nanoparticle is about 100 nm, as measured by DLS analysis.

16. A pharmaceutical formulation comprising the nanoparticle of any of paragraphs 1-15,
wherein the formulation is in a form selected from the group consisting of a solution, a dry powder, a tablet, micelles, colloids, nanodroplets, nano-structured hydrogel, nanocrystals, and a nanosuspension.

17. A method of treating, retarding development of, or preventing development of hyperuricemia in a subject, the method comprising administering to the subject in need thereof an effective amount of the pharmaceutical formulation of paragraph 16 to treat, retard development of, or prevent development of hyperuricemia in the subject.

18. The method of paragraph 17, wherein the method treats, retards development of, or prevents development of one or more diseases or disorders associated with hyperuricemia in the subject,
wherein the disease or disorder is selected from the group consisting of Gout, hypertension, renal disease, Tumor Lysis Syndrome (TLS), and Lesch-Nyhan disease.

19. The method of paragraph 17 or 18, wherein the pharmaceutical formulation is administered to the subject via a route selected from the group consisting of oral administration, intramuscular injection, intravenous injection, sub-cutaneous injection, and intra-articular injection.

20. The method of any one of paragraphs 17-19, wherein the formulation is administered in an amount effective to reduce uric acid in the blood of the subject to an amount less than or equal to 6 mg/dL uric acid in the blood.

21. A composition comprising a gene editing system and a donor sequence encoding one or more uricase enzymes.

22. The composition of paragraph 21, wherein the gene editing system is a CRISPR/Cas system.

23. The composition of paragraph 22, wherein the CRISPR/Cas system comprises (a) a single-guide RNA (sgRNA) that targets the human AAVS1 locus and (b) a Cas nuclease or nickase.

24. The composition of any one of paragraphs 21-23, wherein the one or more uricase enzymes are an ancestral form of a human uricase or a variant thereof.

25. The composition of any one of paragraphs 21-24, wherein one or more uricase enzymes are functional recombinant uricase enzymes each independently having an amino acid sequence of any one or more of SEQ ID NOs:1-8.

26. A method of inducing or increasing uricase enzyme in a subject in need thereof, the method comprising administering to the subject an effective amount of the composition of one of paragraphs 21-25.

27. The method of paragraph 26, wherein the subject has or is at risk of developing hyperuricemia.

28. The method of paragraph 26 or 27, wherein the composition is administered in an amount effective to treat, retard development of, or prevent development of one or more diseases or disorders associated with hyperuricemia in the subject,
wherein the disease or disorder is selected from the group consisting of Gout, hypertension, renal disease, Tumor Lysis Syndrome (TLS), and Lesch-Nyhan disease.

29. A uricase comprising at least one uricase subunit having an amino acid sequence that is at least about 50%, 55%, 60%, 65% 70%, 75%, 80%, 85%, 90%, 95%, 96%, 97%, 98%, 99% or 100% identical to one or more of the respective amino acid sequences of SEQ ID NOs:1-8.

30. The uricase of paragraph 29, wherein the at least one uricase subunit has a cysteine at least at one or more of the following amino acid residues: position 56, position 236, position 68, position 290, position 223, position 130, position 237, position 60, position 13, position 299, position 25, and position 287.

31. The uricase of paragraph 29 or 30, wherein the at least one uricase subunit has a cysteine at least at one or more of amino acid residues selected from position 56, position 236, position 68, position 290, position 223, position 130, position 237, position 60, position 13, position 299, position 25, and position 287 and has improved thermostability compared to a uricase without a cysteine at the corresponding position(s).

32. The uricase of any one of paragraphs 29-31, wherein the at least one uricase subunit has the amino acid sequence of SEQ ID NO:1 and one or more mutations selected from D56C, Q236C, T68C, G290C, A223C, A130C, K237C, G60C, E13C, K299C, M25C, and N287C.

33. The uricase of any one of paragraphs 29-32, wherein the at least one uricase subunit has the amino acid sequence of SEQ ID NO:1 and one or more mutations selected from M25C and N287C.

34. The uricase of any one of paragraphs 29-33, wherein the at least one uricase subunit has the amino acid sequence of SEQ ID NO:8.

35. The uricase of any one of paragraphs 29-34, wherein the uricase is a homo-tetramer or a hetero-tetramer.

36. A pharmaceutical composition comprising the uricase of any one of paragraphs 29-35 and a pharmaceutically acceptable excipient.

37. A uricase comprising at least one uricase subunit having an amino acid sequence that is at least about 50%, 55%, 60%, 65% 70%, 75%, 80%, 85%, 90%, 95%, 96%, 97%, 98%, 99% or 100% identical to one or more of the respective amino acid sequences of SEQ ID NOs:1-8.

38. The uricase of paragraph 37, wherein the uricase is a functional recombinant uricase enzyme molecule.

39. The uricase of paragraph 37 or 38, wherein the at least one uricase subunit has a cysteine at least at one or more of amino acid residues selected from position 56, position 236, position 68, position 290, position 223, position 130, position 237, position 60, position 13, position 299, position 25, and position 287.

40. The uricase of any one of paragraphs 37-39, wherein the at least one uricase subunit has a cysteine at least at one or more of amino acid residues selected from position 56, position 236, position 68, position 290, position 223, position 130, position 237, position 60, position 13, position 299, position 25, and position 287 and has improved thermostability compared to a uricase without a cysteine at the corresponding position(s).

41. The uricase of any one of paragraphs 37-40, wherein the at least one uricase subunit has the amino acid sequence of SEQ ID NO:1 and one or more mutations selected from D56C, Q236C, T68C, G290C, A223C, A130C, K237C, G60C, E13C, K299C, M25C, and N287C.

42. The uricase of any one of paragraphs 37-41, wherein the at least one uricase subunit has the amino acid sequence of SEQ ID NO:1 and one or more mutations selected from M25C and N287C.

43. The uricase of any one of paragraphs 37-42, wherein the at least one uricase subunit has the amino acid sequence of SEQ ID NO:8.

44. The uricase of any one of paragraphs 37-43, wherein the uricase is a homo-tetramer or a hetero-tetramer.

45. A pharmaceutical composition comprising the uricase of any one of paragraphs 1-8 and a pharmaceutically acceptable excipient.

46. A nanoparticle comprising
a plurality of uricases of any one of paragraphs 37-44, wherein at least one of the uricases is a functional recombinant uricase enzyme molecule,
wherein the nanoparticle has a diameter of between about 50 nm and 1,000 nm, inclusive, as determined by Dynamic Light Scattering (DLS) analysis.

47. The nanoparticle of paragraph 46, wherein each of the uricases is a functional recombinant uricase enzyme molecule.

48. The nanoparticle of paragraph 46 or 47, wherein the plurality of uricases each independently have an amino acid sequence of any one or more of SEQ ID NOs:1-8.

49. The nanoparticle of any one of paragraphs 46-48, wherein the nanoparticle comprises a nanostructure consisting of some or all of the functional recombinant uricase enzyme molecules,
wherein the nanostructure is formed by non-covalent interactions between nine or more of the functional recombinant uricase enzyme molecules.

50. The nanoparticle of any one of paragraphs 46-48, wherein the nanoparticle comprises a nanostructure, wherein the nanostructure consists of
(a) some or all of the functional recombinant uricase enzyme molecules; and
(b) one or more cross-linker molecules,
wherein the nanostructure is formed by assembly of nine or more of the functional recombinant uricase enzyme molecules, wherein the one or more cross-linker molecules cross-link two or more of the functional recombinant uricase enzyme molecules within the nanostructure.

51. The nanoparticle of paragraph 49 or 50, wherein the nanostructure is formed by self-assembly of the functional recombinant uricase enzyme molecules.

52. The nanoparticle of any one of paragraphs 46-48, wherein the nanoparticle is a virus-like-particle (VLP), comprising
(a) the functional recombinant uricase enzyme molecules; and
(b) viral capsid proteins,
wherein the functional recombinant uricase enzyme molecules are encapsulated within the viral capsid proteins.

53. The nanoparticle of paragraph 52, wherein the viral capsid proteins are from a virus selected from the group consisting of bacteriophage Q-beta, bacteriophage PP7, and bacteriophage MS.

54. The nanoparticle of paragraph 52 or 53, wherein the VLP encapsulates between one and one hundred functional recombinant uricase enzyme molecules.

55. The nanoparticle of paragraph 54, wherein the VLP encapsulates between ten and thirty functional recombinant uricase enzyme molecules.

56. The nanoparticle of paragraph 55, wherein the VLP encapsulates twenty-five functional recombinant uricase enzyme molecules.

57. The nanoparticle of any one of paragraphs 52-56, wherein the VLP is modified by attachment of one or more moieties to an external face of one or more of the viral capsid proteins.

58. The nanoparticle of paragraph 57, wherein the one or more moieties are independently selected from the group consisting of a protein, a nucleic acid, a carbohydrate, a polymer, a lipid, a small molecule, a secondary nanoparticle, a microparticle, a cell, and a virus.

59. The nanoparticle of paragraph 57 or 58, wherein one or more of the moieties are functional moieties, wherein the functional moieties are independently selected from the group consisting of a targeting moiety, a fluorescent label, a therapeutic agent and a cross-linking agent.

60. The nanoparticle of any one of paragraphs 46-59, wherein the nanoparticle encapsulates one or more additional active agents selected from the group consisting of therapeutic agents, diagnostic agents, nutraceutical agents, and enzyme catalysts.

61. The nanoparticle of any one of paragraphs 46-60, wherein the diameter of the nanoparticle is between about 75 nm and about 150 nm, as measured by DLS analysis.

62. The nanoparticle of any one of paragraphs 46-61, wherein the diameter of the nanoparticle is about 100 nm, as measured by DLS analysis.

63. A pharmaceutical formulation comprising the nanoparticle of any of paragraphs 46-62,
wherein the formulation is in a form selected from the group consisting of a solution, a dry powder, a tablet, micelles, colloids, nanodroplets, nano-structured hydrogel, nanocrystals, and a nanosuspension.

64. A method of treating, retarding development of, or preventing development of hyperuricemia in a subject, the method comprising administering to the subject in need thereof an effective amount of the pharmaceutical formulation of paragraph 63 to treat, retard development of, or prevent development of hyperuricemia in the subject.

65. The method of paragraph 64, wherein the method treats, retards development of, or prevents development of one or more diseases or disorders associated with hyperuricemia in the subject, wherein the disease or disorder is selected from the group consisting of Gout, hypertension, renal disease, Tumor Lysis Syndrome (TLS), and Lesch-Nyhan disease.

66. The method of paragraph 64 or 65, wherein the pharmaceutical formulation is administered to the subject via a route selected from the group consisting of oral administration, intramuscular injection, intravenous injection, sub-cutaneous injection, and intra-articular injection.

67. The method of any one of paragraphs 64-66, wherein the formulation is administered in an amount effective to reduce uric acid in the blood of the subject to an amount less than or equal to 6 mg/dL uric acid in the blood.

68. A composition comprising a gene editing system and a donor sequence encoding one or more uricase enzymes.

69. The composition of paragraph 68, wherein the gene editing system is a CRISPR/Cas system.

70. The composition of paragraph 69, wherein the CRISPR/Cas system comprises (a) a single-guide RNA (sgRNA) that targets the human AAVS1 locus and (b) a Cas nuclease or nickase.

71. The composition of any one of paragraphs 68-70, wherein the one or more uricase enzymes are an ancestral form of a human uricase or a variant thereof.

72. The composition of any one of paragraphs 68-71, wherein one or more uricase enzymes are functional recombinant uricase enzymes each independently having an amino acid sequence that is at least about 50%, 55%, 60%, 65% 70%, 75%, 80%, 85%, 90%, 95%, 96%, 97%, 98%, 99% or 100% identical to one or more of the respective amino acid sequences of SEQ ID NOs:1-8.

73. The composition of paragraph 72, wherein one or more uricase enzymes are functional recombinant uricase enzymes each independently having an amino acid sequence of any one or more of SEQ ID NOs:1-8.

74. A method of inducing or increasing uricase enzyme in a subject in need thereof, the method comprising administering to the subject an effective amount of the composition of one of paragraphs 68-73.

75. The method of paragraph 74, wherein the subject has or is at risk of developing hyperuricemia.

76. The method of paragraph 74 or 75, wherein the composition is administered in an amount effective to treat, retard development of, or prevent development of one or more diseases or disorders associated with hyperuricemia in the subject,
wherein the disease or disorder is selected from the group consisting of Gout, hypertension, renal disease, Tumor Lysis Syndrome (TLS), and Lesch-Nyhan disease.

1. A uricase comprising at least one uricase subunit having an amino acid sequence that is at least about 50%, 55%, 60%, 65% 70%, 75%, 80%, 85%, 90%, 95%, 96%, 97%, 98%, 99% or 100% identical to one or more of the respective amino acid sequences of SEQ ID NOs:1-8.

2. The uricase of claim 1, wherein the uricase is a functional recombinant uricase enzyme molecule.

3. The uricase of claim 1, wherein the at least one uricase subunit has a cysteine at least at one or more of amino acid residues selected from position 56, position 236, position 68, position 290, position 223, position 130, position 237, position 60, position 13, position 299, position 25, and position 287.

4. (canceled)

5. The uricase of claim 1, wherein the at least one uricase subunit has the amino acid sequence of SEQ ID NO:1 and one or more mutations selected from D56C, Q236C, T68C, G290C, A223C, A130C, K237C, G60C, E13C, K299C, M25C, and N287C.

6. (canceled)

7. The uricase of claim 1, wherein the at least one uricase subunit has the amino acid sequence of SEQ ID NO:8.

8. The uricase of claim 1, wherein the uricase is a homo-tetramer or a hetero-tetramer.

9. A pharmaceutical composition comprising the uricase of any one of claim 1, and a pharmaceutically acceptable excipient.

10. A nanoparticle comprising
a plurality of uricases of claim 1, wherein at least one of the uricases is a functional recombinant uricase enzyme molecule,
wherein the nanoparticle has a diameter of between about 50 nm and 1,000 nm, inclusive, as determined by Dynamic Light Scattering (DLS) analysis.

11. The nanoparticle of claim 10, wherein each of the uricases is a functional recombinant uricase enzyme molecule.

12. The nanoparticle of claim 10, wherein the plurality of uricases each independently have an amino acid sequence of any one or more of SEQ ID NOs:1-8.

13. The nanoparticle of claim 10, wherein the nanoparticle comprises a nanostructure consisting of some or all of the functional recombinant uricase enzyme molecules,
wherein the nanostructure is formed by non-covalent interactions between nine or more of the functional recombinant uricase enzyme molecules.

14. The nanoparticle of claim 10, wherein the nanoparticle comprises a nanostructure, wherein the nanostructure consists of
(a) some or all of the functional recombinant uricase enzyme molecules; and
(b) one or more cross-linker molecules,
wherein the nanostructure is formed by assembly of nine or more of the functional recombinant uricase enzyme molecules, wherein the one or more cross-linker molecules cross-link two or more of the functional recombinant uricase enzyme molecules within the nanostructure.

15. (canceled)

16. The nanoparticle of claim 10, wherein the nanoparticle is a virus-like-particle (VLP), comprising
(a) the functional recombinant uricase enzyme molecules; and
(b) viral capsid proteins,
wherein the functional recombinant uricase enzyme molecules are encapsulated within the viral capsid proteins.

17. The nanoparticle of claim 16, wherein the viral capsid proteins are from a virus selected from the group consisting of bacteriophage Q-beta, bacteriophage PP7, and bacteriophage MS.

18. The nanoparticle of claim 16, wherein the VLP encapsulates between one and one hundred functional recombinant uricase enzyme molecules.

19. (canceled)

20. (canceled)

21. The nanoparticle of claim 16, wherein the VLP is modified by attachment of one or more moieties to an external face of one or more of the viral capsid proteins.

22. The nanoparticle of claim 21, wherein the one or more moieties are independently selected from the group consisting of a protein, a nucleic acid, a carbohydrate, a polymer, a lipid, a small molecule, a secondary nanoparticle, a microparticle, a cell, and a virus.

23. The nanoparticle of claim 21, wherein one or more of the moieties are functional moieties, wherein the functional moieties are independently selected from the group consisting of a targeting moiety, a fluorescent label, a therapeutic agent and a cross-linking agent.

24. (canceled)

25. The nanoparticle of claim 10, wherein the diameter of the nanoparticle is between about 75 nm and about 150 nm, as measured by DLS analysis.

26. (canceled)

27. A pharmaceutical formulation comprising the nanoparticle of claim 10,
wherein the formulation is in a form selected from the group consisting of a solution, a dry powder, a tablet, micelles, colloids, nanodroplets, nano-structured hydrogel, nanocrystals, and a nanosuspension.

28. A method of treating, retarding development of, or preventing development of hyperuricemia in a subject, the method comprising administering to the subject in need thereof an effective amount of the pharmaceutical formulation of claim 27 to treat, retard development of, or prevent development of hyperuricemia in the subject.

29. (canceled)

30. (canceled)

31. The method of claim 28, wherein the formulation is administered in an amount effective to reduce uric acid in the blood of the subject to an amount less than or equal to 6 mg/dL uric acid in the blood.

32. A composition comprising a gene editing system and a donor sequence encoding one or more uricase enzymes.

33. The composition of claim 32, wherein the gene editing system is a CRISPR/Cas system.

34. The composition of claim 33, wherein the CRISPR/Cas system comprises (a) a single-guide RNA (sgRNA) that targets the human AAVS1 locus and (b) a Cas nuclease or nickase.

35. The composition of claim 32, wherein the one or more uricase enzymes are an ancestral form of a human uricase or a variant thereof.

36. The composition of claim 32, wherein one or more uricase enzymes are functional recombinant uricase enzymes each independently having an amino acid sequence that is at least about 50%, 55%, 60%, 65% 70%, 75%, 80%, 85%, 90%, 95%, 96%, 97%, 98%, 99% or 100% identical to one or more of the respective amino acid sequences of SEQ ID NOs:1-8.

37. The composition of claim 36, wherein one or more uricase enzymes are functional recombinant uricase enzymes each independently having an amino acid sequence of any one or more of SEQ ID NOs:1-8.

38. A method of inducing or increasing uricase enzyme in a subject in need thereof, the method comprising administering to the subject an effective amount of the composition of claim 32.

39. The method of claim 38, wherein the subject has or is at risk of developing hyperuricemia.

40. (canceled)


SEQUENCE LISTING (22 sequences)

SEQ ID NO: 1 - protein, 304 aa, Homo sapiens
MAHYHNDYKKNDEVEFVRTGYGKDMVKVLHIQRDGKYHSIKEVATSVQLTLSSKKDYLHGDNSDIIPTDTIKNTVHVLAKFKGIKSIEAFAMNICEHFLSSFNHVIRAQVYVEEVPWKRFEKNGVKHVHAFIHTPTGTHFCEVEQMRSGPPVIHSGIKDLKVLKTTQSGFEGFIKDQFTTLPEVKDRCFATQVYCKWRYHQGRDVDFEATWDTVRDIVLEKFAGPYDKGEYSPSVQKTLYDIQVLSLSRVPEIEDMEISLPNIHYFNIDMSKMGLINKEEVLLPLDNPYGKITGTVKRKLSSRL

SEQ ID NO: 2 - protein, 304 aa, Homo sapiens
MAHYHNDYKKNDEVEFVRTGYGKDMVKVLHIQRDGKYHSIKEVATSVQLTLASKKDYLKGDNSDIIPTDTIKNTVHVLAKFKGIKSIEAFAMNICEHFLSSFNHVIRAQVYVEEVPWKRFEKNGVKHVHAFIHTPTGTHRCEVEQMRSGPPVIHSGIKDLKVLKTTQSGFEGFIKDQFTTLPEVKDRVFATQVYCKWRYHQGRDVDFEATWDTVRDIVLEKFAGPYDKGEYSPSVQKTLYDIQVLSLSRVPEIEDMEISLPNIHYFNIDMSKMGLINKEEVLLPLDNPYGKITGTVKRKLSSRL

SEQ ID NO: 3 - protein, 304 aa, Homo sapiens
MAHYHNDYKKNDEVEFVRTGYGKDMVKVLHIQRDGKYHSIKEVATSVQLTLSSKKCYLHGDNSDIIPTDTIKNTVHVLAKFKGIKSIEAFAMNICEHFLSSFNHVIRAQVYVEEVPWKRFEKNGVKHVHAFIHTPTGTHFCEVEQMRSGPPVIHSGIKDLKVLKTTQSGFEGFIKDQFTTLPEVKDRCFATQVYCKWRYHQGRDVDFEATWDTVRDIVLEKFAGPYDKGEYSPSVCKTLYDIQVLSLSRVPEIEDMEISLPNIHYFNIDMSKMGLINKEEVLLPLDNPYGKITGTVKRKLSSRL

SEQ ID NO: 4 - protein, 304 aa, Homo sapiens
MAHYHNDYKKNDEVEFVRTGYGKDMVKVLHIQRDGKYHSIKEVATSVQLTLSSKKDYLHGDNSDIIPCDTIKNTVHVLAKFKGIKSIEAFAMNICEHFLSSFNHVIRAQVYVEEVPWKRFEKNGVKHVHAFIHTPTGTHFCEVEQMRSGPPVIHSGIKDLKVLKTTQSGFEGFIKDQFTTLPEVKDRCFATQVYCKWRYHQGRDVDFEATWDTVRDIVLEKFAGPYDKGEYSPSVQKTLYDIQVLSLSRVPEIEDMEISLPNIHYFNIDMSKMGLINKEEVLLPLDNPYCKITGTVKRKLSSRL

SEQ ID NO: 5 - protein, 304 aa, Homo sapiens
MAHYHNDYKKNDEVEFVRTGYGKDMVKVLHIQRDGKYHSIKEVATSVQLTLSSKKDYLHGDNSDIIPTDTIKNTVHVLAKFKGIKSIEAFAMNICEHFLSSFNHVIRAQVYVEEVPWKRFEKNGVKHVHCFIHTPTGTHFCEVEQMRSGPPVIHSGIKDLKVLKTTQSGFEGFIKDQFTTLPEVKDRCFATQVYCKWRYHQGRDVDFEATWDTVRDIVLEKFCGPYDKGEYSPSVQKTLYDIQVLSLSRVPEIEDMEISLPNIHYFNIDMSKMGLINKEEVLLPLDNPYGKITGTVKRKLSSRL

SEQ ID NO: 6 - protein, 304 aa, Homo sapiens
MAHYHNDYKKNDEVEFVRTGYGKDMVKVLHIQRDGKYHSIKEVATSVQLTLSSKKDYLHCDNSDIIPTDTIKNTVHVLAKFKGIKSIEAFAMNICEHFLSSFNHVIRAQVYVEEVPWKRFEKNGVKHVHAFIHTPTGTHFCEVEQMRSGPPVIHSGIKDLKVLKTTQSGFEGFIKDQFTTLPEVKDRCFATQVYCKWRYHQGRDVDFEATWDTVRDIVLEKFAGPYDKGEYSPSVQCTLYDIQVLSLSRVPEIEDMEISLPNIHYFNIDMSKMGLINKEEVLLPLDNPYGKITGTVKRKLSSRL

SEQ ID NO: 7 - protein, 304 aa, Homo sapiens
MAHYHNDYKKNDCVEFVRTGYGKDMVKVLHIQRDGKYHSIKEVATSVQLTLSSKKDYLHGDNSDIIPTDTIKNTVHVLAKFKGIKSIEAFAMNICEHFLSSFNHVIRAQVYVEEVPWKRFEKNGVKHVHAFIHTPTGTHFCEVEQMRSGPPVIHSGIKDLKVLKTTQSGFEGFIKDQFTTLPEVKDRCFATQVYCKWRYHQGRDVDFEATWDTVRDIVLEKFAGPYDKGEYSPSVQKTLYDIQVLSLSRVPEIEDMEISLPNIHYFNIDMSKMGLINKEEVLLPLDNPYGKITGTVKRCLSSRL

SEQ ID NO: 8 - protein, 304 aa, Homo sapiens
MAHYHNDYKKNDEVEFVRTGYGKDCVKVLHIQRDGKYHSIKEVATSVQLTLSSKKDYLHGDNSDIIPTDTIKNTVHVLAKFKGIKSIEAFAMNICEHFLSSFNHVIRAQVYVEEVPWKRFEKNGVKHVHAFIHTPTGTHFCEVEQMRSGPPVIHSGIKDLKVLKTTQSGFEGFIKDQFTTLPEVKDRCFATQVYCKWRYHQGRDVDFEATWDTVRDIVLEKFAGPYDKGEYSPSVQKTLYDIQVLSLSRVPEIEDMEISLPNIHYFNIDMSKMGLINKEEVLLPLDCPYGKITGTVKRKLSSRL

SEQ ID NO: 9 - RNA, 19 nt, synthetic construct
  source 1..19: mol_type = other RNA
accccacagtggggccact

SEQ ID NO: 10 - RNA, 20 nt, synthetic construct
  source 1..20: mol_type = other RNA
ctagggacaggattggtgac

SEQ ID NO: 11 - RNA, 25 nt, synthetic construct
  source 1..25: mol_type = other RNA
caccgaccccacagtggggccacta

SEQ ID NO: 12 - RNA, 25 nt, synthetic construct
  source 1..25: mol_type = other RNA
aaactagtggccccactgtggggtc

SEQ ID NO: 13 - RNA, 24 nt, synthetic construct
  source 1..24: mol_type = other RNA
caccgtcaccaatcctgtccctag

SEQ ID NO: 14 - RNA, 24 nt, synthetic construct
  source 1..24: mol_type = other RNA
aaacctagggacaggattggtgac

SEQ ID NO: 15 - DNA, 40 bp, synthetic construct
  source 1..40: mol_type = other DNA
atcccggccctaggctcgagatggccagcagcgaggacgt

SEQ ID NO: 16 - DNA, 40 bp, synthetic construct
  source 1..40: mol_type = other DNA
tcaccaatcctgtccctagttaagatacattgatgagttt

SEQ ID NO: 17 - DNA, 40 bp, synthetic construct
  source 1..40: mol_type = other DNA
aaactcatcaatgtatcttaactagggacaggattggtga

SEQ ID NO: 18 - DNA, 40 bp, synthetic construct
  source 1..40: mol_type = other DNA
acgtcctcgctgctggccatctcgagcctagggccgggat

SEQ ID NO: 19 - DNA, 26 bp, synthetic construct
  source 1..26: mol_type = other DNA
ggcagcctgtgctgacccatgcagtc

SEQ ID NO: 20 - skipped

SEQ ID NO: 21 - DNA, 22 bp, synthetic construct
  source 1..22: mol_type = other DNA
aacccttacggcaagatcaccg

SEQ ID NO: 22 - DNA, 20 bp, synthetic construct
  source 1..20: mol_type = other DNA
ccacagttggaggagaatcc